(19) United States
(12) Patent Application Publication
(10) Pub. No.: US 2025/0382619 A1
(43) Pub. Date: Dec. 18, 2025

(54) CANCER THERAPY WITH TGF-BETA-2 AND IRINOTECAN AGENTS

(71) Applicant: GMP BIOTECHNOLOGY LIMITED, Hong Kong (CN)

(72) Inventor: Vuong TRIEU, Agoura Hills, CA (US)

(21) Appl. No.: 19/310,515

(22) Filed: Aug. 26, 2025

Related U.S. Application Data
(63) Continuation of application No. PCT/US2024/017808, filed on Feb. 29, 2024.
(60) Provisional application No. 63/449,286, filed on Mar. 1, 2023.

(51) Int. Cl. C12N 15/113; A61K 31/4745; A61K 45/06; A61P 35/00

(52) U.S. Cl. CPC C12N 15/1136; A61K 31/4745; A61K 45/06; A61P 35/00; C12N 2310/11; C12N 2310/351

(57) ABSTRACT

This invention describes methods for treating or ameliorating the symptoms of cancer in a subject with agents, compositions and regimens designed to promote anti-tumor effects and avoid drug resistance effects. Combinations of active agents can be used including agents for inhibiting or suppressing expression of TGF-β2 in combination with an irinotecan-containing agent, regimen or formulation, for example, a FOLFIRINOX agent, a NALIRIFOX agent, an IRIFOX agent, or an IRINOX agent. Biomarkers can be used to select subjects who benefit from such therapeutics.

### SEQUENCE LISTING

This application includes a sequence listing submitted electronically as an ST.26 file created on Aug. 26, 2025, named 018988-008US1_SL.xml, which is 120,202 bytes in size.

### TECHNICAL FIELD

This invention describes methods for treating or ameliorating the symptoms of cancer in a human or animal subject with agents and compositions designed to promote anti-tumor effects. Exemplary synergistic pharmaceutical therapies include combinations of active agents including agents for inhibiting or suppressing expression of TGF-β2 in combination with an irinotecan-containing agent or formulation. Biomarkers can be used to select subjects who benefit from the agent, use, or methods.

### BACKGROUND

Cancer is a complex pathology involving multiple variant cellular pathways. Because of this complexity, it has been difficult to find effective therapeutics that can have sufficient anti-tumor effects in various cancers. Making matters worse, cancer cells and malignancies can develop resistance to various agents and drugs and show lack of patient response.

Irinotecan is an example of a conventional therapy that has been used against various malignancies. Drawbacks of conventional therapies such as compositions containing irinotecan include lack of sufficient efficacy over a range of cancers. Further drawbacks include the development of resistance to irinotecan which can limit successful treatment outcomes. Additional drawbacks of the use of compositions containing irinotecan include cross resistance to other anti-cancer drugs. Moreover, not all cancer patients respond robustly to a therapy based on irinotecan and must be given palliative care instead. Such drawbacks can reduce the available treatment options.

What is needed to overcome these difficulties are methods and uses which employ combinations of compositions and agents to increase efficacy against malignancies and avoid resistance effects.

There is an urgent need for compositions, agents, uses, and methods with different agents in combination having significant anti-tumor effects. There is a further need for improved guidance in the use of such compositions and agents by using appropriate clinically-validated biomarkers to provide synergistic improvement in efficacy.

### BRIEF SUMMARY

This invention describes methods for treating or ameliorating the symptoms of cancer in a human or animal subject with agents and compositions designed to promote anti-tumor effects over a range of different cancers and avid drug resistance effects.

Embodiments of this disclosure include agents and methods for treating or ameliorating the symptoms of cancer in a human or animal subject in need with an agent for inhibiting or suppressing expression of TGF-β2.

Synergistic pharmaceutical therapies of this invention include combinations of active agents such as agents for inhibiting or suppressing expression of TGF-β2 with agents such as irinotecan and irinotecan-containing formulations.

In some embodiments, biomarkers can be used to select subjects who benefit from the agent, use, or methods.

In further embodiments, the agents and compositions can be used in combination with chemotherapy, radiation therapy and other standard-of-care therapies.

This disclosure includes uses of compositions containing an agent for inhibiting or suppressing expression of TGF-β2 in the preparation of a medicament for treating or ameliorating the symptoms of cancer in a human or animal subject.

This invention contemplates agents, uses, and methods for treating or ameliorating the symptoms of a range of different cancers, including pancreatic cancer, colorectal cancer, stomach cancer, cervical cancer, lung cancer, melanoma, skin cancer, breast cancer, prostate cancer, kidney cancer, ovarian cancer, thymus cancer, and multiple myeloma.

Embodiments of this invention include the following:

An antisense agent for inhibiting or suppressing expression of TGF-β2 in combination with an agent comprising irinotecan for use in treating or ameliorating the symptoms of cancer in a subject.

Use of an antisense agent for inhibiting or suppressing expression of TGF-β2 in the preparation of a medicament for treating or ameliorating the symptoms of a cancer in a subject in combination with an agent comprising irinotecan.

A method for treating or ameliorating the symptoms of cancer in a subject in need, the method comprising:

administering a therapeutically effective amount of an antisense agent for inhibiting or suppressing expression of TGF-β2 to the subject; and

administering a therapeutically effective amount of an agent comprising irinotecan to the subject.

The agent, use or method above, wherein the agent comprising irinotecan is a FOLFIRINOX agent, a NALIRIFOX agent, an IRIFOX agent, or an IRINOX agent.

The agent, use or method above, wherein the agent comprising irinotecan comprises a nano-sized particle, a nano-sized micelle, a nano-sized liposome, or a nano-sized exosome.

The agent, use or method above, wherein the agent for inhibiting or suppressing expression of TGF-β2 and the agent comprising irinotecan are administered concurrently, simultaneously, sequentially, or separately in time.

The agent, use or method above, wherein the agent for inhibiting or suppressing expression of TGF-β2 and the agent comprising irinotecan are administered separately or in combination by injection or infusion.

The agent, use or method above, wherein the cancer is a pancreatic cancer, a colorectal cancer, a stomach cancer, a cervical cancer, a lung cancer, melanoma, a skin cancer, a breast cancer, a prostate cancer, a kidney cancer, an ovarian cancer, a thymus cancer, and multiple myeloma.

The agent, use or method above, wherein the agent for inhibiting or suppressing expression of TGF-β2 is a TGF-β2-specific antisense oligonucleotide complementary to a TGF-β2 transcript and 15-30 nucleotides in length.

The agent, use or method above, wherein the agent for inhibiting or suppressing expression of TGF-β2 is a TGF-β2-specific antisense oligonucleotides complementary to a TGF-β2 pre-RNA, pre-mRNA or mRNA and 18-21 nucleotides in length.

The agent, use or method above, wherein the agent for inhibiting or suppressing expression of TGF-β2 is one or more TGF-β2-specific antisense oligonucleotides complementary to a TGF-β2 transcript as in Table 1 and chemically-modified variants thereof, LNA variants thereof, gapmer variants thereof, and any combination or pooling thereof.

The agent, use or method above, wherein the TGF-β2-specific antisense oligonucleotides have no more than one or two mismatches as compared to a target human TGF-β2.

The agent, use or method above, wherein the TGF-β2-specific antisense oligonucleotides reduce a TGF-β2 transcript level by at least 60%, or at least 70%, or at least 80%, or at least 90%.

The agent, use or method above, wherein the TGF-β2-specific antisense oligonucleotides reduce any TGF-β1 transcript level and any TGF-β3 transcript level by less than 10%, or less than 5%, or less than 1%.

The agent, use or method above, wherein the TGF-β2-specific antisense oligonucleotides have one or more nucleotides chemically modified as a phosphorothioate internucleoside linkage, a methoxypropylphosphonate internucleoside linkage, an aminophosphoro linkage to a morpholino group, a 2′-OMe ribose group, a 2′-MOE methoxyethyl ribose group, a 2′-4′ constrained methoxyethyl bicyclic ribose group, a 2′-4′ constrained ethyl bicyclic ribose group, an LNA ribose group, a 2′-F ribose group, or a 5-methylcytodine base.

The agent, use or method above, wherein the antisense agent is conjugated to a polyethylene glycol, a lipid, or a triantenarry N-acteyl-galactosamine.

The agent, use or method above, wherein the agent for inhibiting or suppressing expression of TGF-β2 comprises a carrier of sterile water for injection, saline, isotonic saline, or a combination thereof, which may be the same or different for each agent.

The agent, use or method above, wherein the agent for inhibiting or suppressing expression of TGF-β2 is substantially free of excipients.

The agent, use or method above, wherein the agent for inhibiting or suppressing expression of TGF-β2 is stable in a carrier substantially free of excipients for at least 14 days at 37° C.

The agent, use or method above, wherein patients are selected who have previously been treated with any agent comprising irinotecan.

The agent, use or method above, comprising using one or more biomarkers to select subjects who benefit from the agent, use or method.

The agent, use or method above, wherein patients are selected who have an elevated level of TGF-β2.

The agent, use or method above, wherein the one or more biomarkers are a level of a tumor mutation burden (TMB), a level of a tumor neoantigen, a level of clinical hypoxia, or a combination thereof.

The agent, use or method above, wherein the one or more biomarkers are a level of a tumor mutation burden, a level of a reduced neoantigen load determined in a tumor microenvironment, a level of a reduced macrophage determined in a tumor microenvironment, a level of a reduced mesenchymal stem cell MSC determined in a tumor microenvironment, a level of a type 2 T-helper cell Th2 determined in a tumor microenvironment, and a combination thereof.

The method, agent or use above, wherein the subject upon the administration or use has an improved level of at least one of the one or more biomarkers as compared to a level found in a healthy patient.

The agent, use or method above, wherein the administration or use decreases mortality rate at month 6, 12, 18, 24, 30, or 36.

The agent, use or method above, wherein the administration or use increases survival rate at month 6, 12, 18, 24, 30, or 36.

The agent, use or method above, wherein the administration or use increases health-related quality of life (HRQOL) by 15%, or 25%, or 35%, or 45%.

The agent, use or method above, in combination with any one or more medicaments comprising a targeted cancer drug, a cancer growth blocker, an EGFR inhibitor, and combinations thereof.

The agent, use or method above, in combination with any one or more medicaments selected from bevacizumab, everolimus, belzutifan, dabrafenib, trametinib, and combinations thereof.

The agent, use or method above, in combination with any one or more medicaments which are cancer growth blockers selected from an angiogenesis inhibitor, a histone deacetylase inhibitor, a hedgehog blocker, an mTOR inhibitor, a p53 inhibitor, a PARP inhibitor, a proteasome inhibitor, a tyrosine kinase inhibitor, and combinations thereof.

The agent, use or method above, in combination with any one or more medicaments which are EGFR inhibitors selected from erlotinib, gefitinib, afatinib, osimertinib, dacomitininb, and combinations thereof.

The agent, use or method above, in combination with a standard of care treatment for cancer.

The agent, use or method above, in combination with radiation therapy or electric field therapy.

A kit for treating or ameliorating the symptoms of cancer, the kit comprising: a therapeutically effective amount of an antisense agent for inhibiting or suppressing expression of TGF-β2; and a therapeutically effective amount of an agent comprising irinotecan.

The kit above, wherein the cancer is a pancreatic cancer, a colorectal cancer, a stomach cancer, a cervical cancer, a lung cancer, melanoma, a skin cancer, a breast cancer, a prostate cancer, a kidney cancer, an ovarian cancer, a thymus cancer, and multiple myeloma.

The kit above, wherein the agent comprising irinotecan is a FOLFIRINOX agent, a NALIRIFOX agent, an IRIFOX agent, or an IRINOX agent.

The kit above, wherein the agent comprising irinotecan comprises a nano-sized particle, a nano-sized micelle, a nano-sized liposome, or a nano-sized exosome.

The kit above, wherein the agent for inhibiting or suppressing expression of TGF-β2 and the agent comprising irinotecan are administered concurrently, simultaneously, sequentially, or separately in time.

The kit above, wherein the agent for inhibiting or suppressing expression of TGF-β2 and the agent comprising irinotecan are administered separately or in combination by injection or infusion.

The kit above, wherein the agent for inhibiting or suppressing expression of TGF-β2 is a TGF-β2-specific antisense oligonucleotide complementary to a TGF-β2 transcript and 15-30 nucleotides in length.

The kit above, wherein the agent for inhibiting or suppressing expression of TGF-β2 is a TGF-β2-specific antisense oligonucleotides complementary to a TGF-β2 pre-RNA, pre-mRNA or mRNA and 18-21 nucleotides in length.

The kit above, wherein the agent for inhibiting or suppressing expression of TGF-2 is one or more TGF-β2-specific antisense oligonucleotides complementary to a TGF-β2 transcript as given in Table 1.

The kit above, wherein the TGF-β2-specific antisense oligonucleotides have no more than one or two mismatches as compared to a target human TGF-β2.

The kit above, wherein the TGF-2-specific antisense oligonucleotides reduce a TGF-β2 transcript level by at least 60%, or at least 70%, or at least 80%, or at least 90%.

The kit above, wherein the TGF-β2-specific antisense oligonucleotides reduce any TGF-β1 transcript level and any TGF-β3 transcript level by less than 10%, or less than 5%, or less than 1%.

The kit above, wherein the TGF-β2-specific antisense oligonucleotides have one or more nucleotides chemically modified as a phosphorothioate internucleoside linkage, a methoxypropylphosphonate internucleoside linkage, an aminophosphoro linkage to a morpholino group, a 2′-OMe ribose group, a 2′-MOE methoxyethyl ribose group, a 2′-4′ constrained methoxyethyl bicyclic ribose group, a 2′-4′ constrained ethyl bicyclic ribose group, an LNA ribose group, a 2′-F ribose group, or a 5-methylcytodine base.

The kit above, wherein the antisense agent is conjugated to a polyethylene glycol, a lipid, or a triantenarry N-acteyl-galactosamine.

The kit above, wherein the agent for inhibiting or suppressing expression of TGF-β2 comprises a carrier of sterile water for injection, saline, isotonic saline, or a combination thereof, which may be the same or different for each agent.

The kit above, wherein the agent for inhibiting or suppressing expression of TGF-β2 is substantially free of excipients.

The kit above, wherein the agent for inhibiting or suppressing expression of TGF-2 is stable in a carrier substantially free of excipients for at least 14 days at 37° C.

### BRIEF DESCRIPTION OF THE DRAWINGS

FIG. 1 shows highly significant improvement in overall survival for pancreatic cancer patients with reduced TGF-β2.

FIG. 2 shows highly significant improvement in overall survival for pancreatic cancer patients with reduced TGF-β2.

FIG. 3 shows highly significant improvement in overall survival for pancreatic cancer patients with increased hypoxia.

FIG. 4 shows highly significant improvement in overall survival for pancreatic cancer patients with reduced TMB.

FIG. 5 shows highly significant and surprising improvement in overall survival for pancreatic cancer patients with reduced TGF-β2 and treatment with irinotecan.

FIG. 6 shows highly significant and surprising improvement in overall survival for pancreatic cancer patients with reduced TGF-β2 and treatment with irinotecan.

FIG. 7 shows highly significant and surprising improvement in overall survival for pancreatic cancer patients with reduced TGF-β2 and treatment with FOFIR irinotecan agent.

FIG. 8 shows highly significant improvement in overall survival for pancreatic cancer patients with reduced TGF-β2 where patients were selected with reduced macrophage.

FIG. 9 shows highly significant improvement in overall survival for pancreatic cancer patients with reduced TGF-β2 where patients were selected with a combination of reduced neoantigen load determined in a tumor microenvironment and reduced macrophage.

### DETAILED DESCRIPTION OF THE DISCLOSURE

This invention relates to methods, compositions, agents and therapeutic uses thereof for treating or ameliorating the symptoms of cancer in a human or animal subject with agents and compositions designed to promote anti-tumor effects over a range of different cancers and avoid drug resistance effects.

Examples of synergistic pharmaceutical therapies of this invention include combinations of active agents such as agents for inhibiting or suppressing expression of TGF-β2 with agents such as irinotecan and irinotecan-containing formulations.

Embodiments of this invention include methods, agents and therapeutic uses thereof for treating or ameliorating symptoms of oncological disease in which agents may be administered concurrently, simultaneously, sequentially, or separately in time.

In certain embodiments, a highly stable formulation of one or more anti-TGF-β2 agents may be used for oncological disease in combination with one or more irinotecan-containing agents, in which the anti-TGF-β2 agents and the irinotecan-containing agents are used concurrently, simultaneously, sequentially, or separately in time.

In some embodiments, one or more biomarkers can be used to select subjects who benefit from the method, agent or use. Combinations of therapeutic agents disclosed herein guided by biomarker selection can provide surprising improvement in patient overall survival.

Therapeutic compositions of this disclosure can also be used in combination with chemotherapy and other standard-of-care therapies.

In some embodiments, this invention contemplates a combination of TGF-β2 inhibiting agents with agents such as irinotecan and irinotecan formulations, which can be guided by biomarkers.

This invention recognizes that overexpression of TGF-β2 is a useful indicator for avoiding a cascade of downstream effects and poor outcomes in oncological disease.

In some embodiments, subjects can be selected for therapy based on TGF-β2 as biomarker to provide improved outcomes. A subject may be selected when expression of TGF-β2 is elevated.

In some embodiments, detection of certain biomarkers can be utilized as a guide to select subjects for therapy which comprises a combination of TGF-β2 inhibitors and various anti-cancer agents.

In some embodiments, this invention includes antisense oligonucleotides as agents to selectively target and inhibit or suppress expression of TGF-β2 in a patient. Such agents are surprisingly superior to agents targeted to TGF-β1 or TGF-β3 for outcomes in oncological disease.

In some embodiments, the combination of a TGF-β2 antisense inhibitor with anti-cancer agents based on irinotecan and/or other agents can be surprisingly effective.

A hallmark of improved efficacy and avoidance of drug resistance effects is improvement in patient overall survival (OS).

In further embodiments, methods for treating or ameliorating the symptoms of cancer in a human subject or animal subject in need include administering a therapeutically sufficient amount of a pharmaceutical composition comprising an agent for inhibiting or suppressing expression of TGF-β2 to the subject, and administering a therapeutically sufficient amount of a pharmaceutical composition comprising an agent such as irinotecan or an irinotecan-containing formulation, where the subject may be selected using a TGF-β2 biomarker, and where the subject may be selected when expression of TGF-β2 is elevated.

In additional embodiments, this invention includes an antisense agent for inhibiting or suppressing expression of TGF-β2 in combination with an agent comprising irinotecan for use in treating or ameliorating the symptoms of cancer in a subject, where the subject is selected when expression of TGF-β2 is elevated.

In certain embodiments, this invention includes uses of an antisense agent for inhibiting or suppressing expression of TGF-β2 in the preparation of a medicament for treating or ameliorating the symptoms of a cancer in a subject in combination with an agent comprising irinotecan, where the subject is selected when expression of TGF-β2 is elevated.

In some embodiments, this invention includes methods for treating or ameliorating the symptoms of cancer in a subject in need, the method comprising administering a therapeutically effective amount of an antisense agent for inhibiting or suppressing expression of TGF-β2 to the subject, and administering a therapeutically effective amount of an agent comprising irinotecan to the subject, where the subject is selected when expression of TGF-β2 is elevated.

Therapies of this invention may be applied for a range of different cancers, including pancreatic cancer, colorectal cancer, stomach cancer, cervical cancer, lung cancer, melanoma, skin cancer, breast cancer, prostate cancer, kidney cancer, ovarian cancer, thymus cancer, and multiple myeloma.

### Methods and Compositions for Cancer

Many cancers such as pancreatic cancer, melanoma, and others present patients with high levels of expression of TGF-β2. Agents for inhibiting or suppressing expression of TGF-β2 can be effective in treating these cancer types. For example, in patients with pancreatic cancer, overall survival time can be more than doubled from 15 months for high TGF-β2 patients to 37 months for low TGF-β2 patients.

This invention includes methods for treating or ameliorating the symptoms of cancer in a human or mammalian subject in need, by administering a therapeutically sufficient amount of a pharmaceutical composition comprising an agent for inhibiting or suppressing expression of TGF-β2 to the subject in combination with additional anti-cancer agents such as irinotecan or an irinotecan-containing agent.

In certain embodiments, this invention includes agents for inhibiting or suppressing expression of TGF-β2 in combination with an agent such as irinotecan or an irinotecan-containing agent, such as a FOLFIRI agent, or an IRIFOX agent, for use in treating or ameliorating the symptoms of cancer in a human or mammalian subject.

This invention further contemplates uses of a composition comprising an agent for inhibiting or suppressing expression of TGF-β2 in the preparation of a medicament for treating or ameliorating the symptoms of a cancer in a subject in combination with agents such as irinotecan or an irinotecan-containing formulation, such as a FOLFIRINOX agent, a NALIRIFOX agent, an IRIFOX agent, or an IRINOX agent.

Therapies of this invention using one or more agents for inhibiting or suppressing expression of TGF-β2 may be used against pancreatic cancer, colorectal cancer, stomach cancer, cervical cancer, lung cancer, melanoma, skin cancer, breast cancer, prostate cancer, kidney cancer, ovarian cancer, thymus cancer, and multiple myeloma.

A pharmaceutical composition of this disclosure may include an agent for inhibiting or suppressing expression of TGF-β2, to be administered in a therapeutically sufficient amount to the subject.

As used herein, the term “agent” can refer to a regimen using one or more active compounds, or a composition containing one or more active compounds and a carrier, and/or a solvent, and/or any number of excipients. In a regimen, active agents or formulations thereof may be administered concurrently, simultaneously, sequentially, or separately in time. In some embodiments, the composition may be a pharmaceutical composition. In certain embodiments, the composition may be a pharmaceutical composition containing a therapeutically effective amount of one or more active compounds. Some examples of excipients are given in Remington's Pharmaceutical Sciences, Mack Publishing Co., Easton, Pa. 1975, and Liberman, H. A. and Lachman, L., Eds., Pharmaceutical Dosage Forms, Marcel Decker, New York, N.Y., 1980. Methods for determining a therapeutically effective amount of an agent are known in the art.

In some embodiments, this invention includes an antisense agent for suppressing expression of TGF-β2 in combination with an agent or regimen comprising irinotecan for use in treating or ameliorating the symptoms of cancer in a subject.

In certain embodiments, this invention includes an antisense agent for suppressing expression of TGF-β2 for use in combination with an agent or regimen comprising irinotecan for use in treating or ameliorating the symptoms of cancer in a subject.

As used herein, the term “an agent comprising irinotecan” can refer to a regimen comprising irinotecan. An agent or regimen comprising irinotecan can include or employ any form of irinotecan.

As used herein, the term a kit comprising “an agent comprising irinotecan” can refer to kit composed of active agents or formulations thereof for use in a regimen.

### Human TGF-β2-Specific Phosphorothioate Antisense Oligodeoxynucleotide Agents

An antisense oligonucleotide (ASO) can be a single-stranded deoxyribonucleotide, which may be complementary to an mRNA target. The antisense therapy may downregulate a molecular target, which may be achieved by induction of RNase H endonuclease activity that cleaves the RNA-DNA heteroduplex with a significant reduction of the target gene translation. Other ASO mechanisms can include inhibition of 5′ cap formation, alteration of splicing process such as splice-switching, and steric hindrance of ribosomal activity.

Antisense therapeutic strategies can utilize single-stranded DNA oligonucleotides that inhibit protein production by mediating the catalytic degradation of a target mRNA, or by binding to sites on mRNA needed for translation. Antisense oligonucleotides can be designed to target the viral RNA genome or viral transcripts. Antisense oligonucleotides can provide an approach for identifying potential targets, and therefore represent potential therapeutics.

Antisense oligonucleotides can be small synthetic pieces of single-stranded DNA that may be 15-30 nucleotides in length. An ASO may specifically bind to a complementary DNA/RNA sequence by Watson-Crick hybridization and once bound to the target RNA, inhibit the translational processes either by inducing cleavage mechanisms or by inhibiting mRNA maturation. An ASO may selectively inhibit gene expression with specificity. Chemical modifications of DNA or RNA can be used to increase stability.

For example, modifications can be introduced in the phosphodiester bond, the sugar ring, and the backbone. ASO antiviral agents may block translational processes either by (i) ribonuclease H (RNAse H) or RNase P mediated cleavage of mRNA or (ii) by sterically (non-bonding) blocking enzymes that are involved in the target gene translation. Human TGF-2-specific phosphorothioate antisense oligodeoxynucleotide, for example OT-101 which is AP 12009 Trabedersen SEQ ID NO: 1, can be used to reduce the level of TGF-β2 protein in malignancies, and thereby delay the progression of disease.

Antisense oligodeoxynucleotides are short strings of DNA that are designed to downregulate gene expression by interfering with the translation of a specific encoded protein at the mRNA level. OT-101 is a synthetic 18-mer phosphorothioate oligodeoxynucleotide (S-ODN) where all 3′-5′ linkages are modified to phosphorothioates. The molecular formula is C₁₇₇H₂₀₈N₆₀Na₁₇O₉₄P₁₇S₁₇ and the molecular weight 6,143 g/mol. OT-101 was designed to be complementary to a specific sequence of human TGF-β2 mRNA following expression of the gene.

Antisense oligodeoxynucleotides are short strings of DNA that are designed to downregulate gene expression by interfering with the translation of a specific encoded protein at the mRNA level. For example, SEQ ID NO: 1 (OT-101) is a synthetic 18-mer phosphorothioate oligodeoxynucleotide (S-ODN) in which a non-bridging oxygen of each phosphate moiety is substituted by a sulfur atom. OT-101 is complementary to a specific sequence of human TGF-β2 mRNA from expression of the gene. OT-101 can be an RNA therapeutic designed to abrogate the immunosuppressive actions of TGF-β2 and reduce the level of TGF-2 in malignancies, to treat or ameliorate the symptoms of cancer, or delay the progression of disease.

A target TGF-β2 mRNA can be NCBI Reference Sequence: NM_003238.3 of sequence length 5,882 bp. A target region for TGF-β2 mRNA can be the protein coding sequence from reference 1,369 to 2,613.

Examples of agents of this disclosure for inhibiting or suppressing expression of TGF-β2 include TGF-β2-specific antisense oligonucleotides given in SEQ ID NOs: 1-136 in Table 1.

TABLE 1
TGF-β2-specific antisense oligonucleotides

| SEQ ID | | |
|---|---|---|
| NO: | Ref. | ANTISENSE SEQUENCE |
| 1 | 1369-2613 | CGGCATGTCT ATTTTGTA |
| 2 | — | GTTCGTTTAG AGAACAGATC |
| 3 | — | TAAAGTTCGT TTAGAGAACA G |
| 4 | — | AGCCCTGTAT ACGAC |
| 5 | — | GTAGGTAAAA ACCTAATAT |
| 6 | — | CGTTTAGAGA ACAGATCTAC |
| 7 | — | CATTGTAGAT GTCAAAAGCC |
| 8 | — | CTCCCTCATG GTGGCAGTTG A |
| 9 | 178-195 | TTTGTTCCTG GATGACTC |
| 10 | 179-196 | GTTTGTTCCT GGATGACT |
| 11 | 180-197 | AGTTTGTTCC TGGATGAC |
| 12 | 181-198 | CAGTTTGTTC CTGGATGA |
| 13 | 182-199 | TCAGTTTGTT CCTGGATG |
| 14 | 183-200 | CTCAGTTTGT TCCTGGAT |
| 15 | 716-733 | TGTGTGTGTG TGCGTGTG |
| 16 | 717-734 | GTGTGTGTGT GTGCGTGT |
| 17 | 718-735 | TGTGTGTGTG TGTGCGTG |
| 18 | 719-736 | GTGTGTGTGT GTGTGCGT |
| 19 | 720-737 | TGTGTGTGTG TGTGTGCG |
| 20 | 721-738 | GTGTGTGTGT GTGTGTGC |
| 21 | 722-739 | TGTGTGTGTG TGTGTGTG |
| 22 | 723-740 | GTGTGTGTGT GTGTGTGT |
| 23 | 724-741 | TGTGTGTGTG TGTGTGTG |
| 24 | 725-742 | GTGTGTGTGT GTGTGTGT |
| 25 | 726-743 | TGTGTGTGTG TGTGTGTG |
| 26 | 727-744 | GTGTGTGTGT GTGTGTGT |
| 27 | 728-745 | TGTGTGTGTG TGTGTGTG |
| 28 | 729-746 | GTGTGTGTGT GTGTGTGT |
| 29 | 730-747 | CGTGTGTGTG TGTGTGTG |
| 30 | 731-748 | GCGTGTGTGT GTGTGTGT |
| 31 | 732-749 | TGCGTGTGTG TGTGTGTG |
| 32 | 733-750 | GTGCGTGTGT GTGTGTGT |
| 33 | 971-988 | AGTGGCGGAT CTGAACTC |
| 34 | 972-989 | GAGTGGCGGA TCTGAACT |
| 35 | 1430-1447 | GAGTGTGCTG CAGGTAGA |
| 36 | 1432-1449 | TCGAGTGTGC TGCAGGTA |
| 37 | 1433-1450 | ATCGAGTGTG CTGCAGGT |
| 38 | 1570-1587 | GTGCTGTTGT AGATGGAA |
| 39 | 1571-1588 | GGTGCTGTTG TAGATGGA |
| 40 | 1572-1589 | TGGTGCTGTT GTAGATGG |
| 41 | 1573-1590 | CTGGTGCTGT TGTAGATG |
| 42 | 1574-1591 | CCTGGTGCTG TTGTAGAT |
| 43 | 1575-1592 | CCCTGGTGCT GTTGTAGA |
| 44 | 1576-1593 | TCCCTGGTGC TGTTGTAG |
| 45 | 1577-1594 | GTCCCTGGTG CTGTTGTA |
| 46 | 1578-1595 | AGTCCCTGGT GCTGTTGT |
| 47 | 1895-1912 | CTGGGTTGGA GATGTTAA |
| 48 | 1896-1913 | GCTGGGTTGG AGATGTTA |
| 49 | 1897-1914 | CGCTGGGTTG GAGATGTT |
| 50 | 1900-1917 | TAGCGCTGGG TTGGAGAT |
| 51 | 1903-1920 | ATGTAGCGCT GGGTTGGA |
| 52 | 1945-1962 | AGCCATTCGC CTTCTGCT |
| 53 | 1994-2011 | GTCTTTATGG TGAAGCCA |
| 54 | 1995-2012 | TGTCTTTATG GTGAAGCC |
| 55 | 1996-2013 | CTGTCTTTAT GGTGAAGC |
| 56 | 1997-2014 | CCTGTCTTTA TGGTGAAG |
| 57 | 2000-2017 | GTTCCTGTCT TTATGGTG |
| 58 | 2001-2018 | GGTTCCTGTC TTTATGGT |
| 59 | 2002-2019 | AGGTTCCTGT CTTTATGG |
| 60 | 2003-2020 | CAGGTTCCTG TCTTTATG |
| 61 | 2004-2021 | CCAGGTTCCT GTCTTTAT |
| 62 | 2183-2200 | GGTCTTCCCA CTGTTTTT |
| 63 | 2194-2211 | AGGAGATGTG GGGTCTTC |
| 64 | 2195-2212 | CAGGAGATGT GGGGTCTT |
| 65 | 2241-2258 | GGTTGGTCTG TTGTGACT |
| 66 | 2242-2259 | CGGTTGGTCT GTTGTGAC |
| 67 | 2243-2260 | CCGGTTGGTC TGTTGTGA |
| 68 | 2522-2539 | GAGAATGGTT AGAGGTTC |
| 69 | 2525-2542 | GTAGAGAATG GTTAGAGG |
| 70 | 2542-2559 | GGTGTTTTGC CAATGTAG |
| 71 | 2543-2560 | GGGTGTTTTG CCAATGTA |
| 72 | 2544-2561 | TGGGTGTTTT GCCAATGT |
| 73 | 2666-2683 | CATCATCGTT GTCGTCGT |
| 74 | 2979-2996 | GAACGGTACG TACAGCAA |
| 75 | 2980-2997 | GGAACGGTAC GTACAGCA |
| 76 | 2981-2998 | AGGAACGGTA CGTACAGC |
| 77 | 2982-2999 | TAGGAACGGT ACGTACAG |
| 78 | 2983-3000 | ATAGGAACGG TACGTACA |
| 79 | 2984-3001 | GATAGGAACG GTACGTAC |
| 80 | 2985-3002 | GGATAGGAAC GGTACGTA |
| 81 | 2986-3003 | GGGATAGGAA CGGTACGT |
| 82 | 3029-3046 | GGGTGCCTAT TGCATAGC |
| 83 | 3030-3047 | AGGGTGCCTA TTGCATAG |
| 84 | 3031-3048 | AAGGGTGCCT ATTGCATA |
| 85 | 3032-3049 | GAAGGGTGCC TATTGCAT |
| 86 | 3033-3050 | GGAAGGGTGC CTATTGCA |
| 87 | 3035-3052 | TGGGAAGGGT GCCTATTG |
| 88 | 3036-3053 | ATGGGAAGGG TGCCTATT |
| 89 | 3037-3054 | AATGGGAAGG GTGCCTAT |
| 90 | 3038-3055 | GAATGGGAAG GGTGCCTA |
| 91 | 3039-3056 | AGAATGGGAA GGGTGCCT |
| 92 | 3040-3057 | AAGAATGGGA AGGGTGCC |
| 93 | 3041-3058 | TAAGAATGGG AAGGGTGC |
| 94 | 3042-3059 | GTAAGAATGG GAAGGGTG |
| 95 | 3043-3060 | AGTAAGAATG GGAAGGGT |
| 96 | 3044-3061 | GAGTAAGAAT GGGAAGGG |
| 97 | 3259-3276 | CAGACTTTCT CGGTCATA |
| 98 | 3260-3277 | GCAGACTTTC TCGGTCAT |
| 99 | 3261-3278 | TGCAGACTTT CTCGGTCA |
| 100 | 3262-3279 | ATGCAGACTT TCTCGGTC |
| 101 | 3263-3280 | AATGCAGACT TTCTCGGT |
| 102 | 3264-3281 | TAATGCAGAC TTTCTCGG |
| 103 | 4281-4298 | GACCTGGACT TTTTTCCC |
| 104 | 4282-4299 | TGACCTGGAC TTTTTTCC |
| 105 | 4283-4300 | CTGACCTGGA CTTTTTTC |
| 106 | 4284-4301 | GCTGACCTGG ACTTTTTT |
| 107 | 4467-4484 | CTGCAATGAT GTGGCAAA |
| 108 | 4468-4485 | TCTGCAATGA TGTGGCAA |
| 109 | 4469-4486 | TTCTGCAATG ATGTGGCA |
| 110 | 4470-4487 | CTTCTGCAAT GATGTGGC |
| 111 | 5062-5079 | GCTGCCCACT TGCATACT |
| 112 | 5569-5586 | GTTGGCAGAA CATAGAAC |
| 113 | 5570-5587 | CGTTGGCAGA ACATAGAA |
| 114 | 5571-5588 | GCGTTGGCAG AACATAGA |
| 115 | 5616-5633 | ATGGGGCTAC AGGGGATA |
| 116 | 5617-5634 | TATGGGGCTA CAGGGGAT |
| 117 | 5618-5635 | TTATGGGGCT ACAGGGGA |
| 118 | 5620-5637 | AGTTATGGGG CTACAGGG |
| 119 | 5621-5638 | AAGTTATGGG GCTACAGG |
| 120 | 5622-5639 | CAAGTTATGG GGCTACAG |
| 121 | 5623-5640 | CCAAGTTATG GGGCTACA |
| 122 | 5624-5641 | TCCAAGTTAT GGGGCTAC |
| 123 | 5625-5642 | ATCCAAGTTA TGGGGCTA |
| 124 | 5626-5643 | TATCCAAGTT ATGGGGCT |
| 125 | 5627-5644 | CTATCCAAGT TATGGGGC |
| 126 | 5759-5776 | ATTGGAGGAA ATAGGGTG |
| 127 | 5783-5800 | GTCTTGTAGG TAGCAGCC |
| 128 | 5784-5801 | GGTCTTGTAG GTAGCAGC |
| 129 | 5785-5802 | TGGTCTTGTA GGTAGCAG |
| 130 | 5786-5803 | CTGGTCTTGT AGGTAGCA |
| 131 | 5787-5804 | TCTGGTCTTG TAGGTAGC |
| 132 | 5788-5805 | GTCTGGTCTT GTAGGTAG |
| 133 | 5789-5806 | AGTCTGGTCT TGTAGGTA |
| 134 | 5790-5807 | GAGTCTGGTC TTGTAGGT |
| 135 | 5791-5808 | GGAGTCTGGT CTTGTAGG |
| 136 | 5792-5809 | AGGAGTCTGG TCTTGTAG |

The sequences of Table 1 can be chemically-modified to provide active variants thereof, LNA variants thereof, as well as gapmer variants thereof, as known in the art. The sequences of Table 1 can be used in any combination as active agents, such as pooling combinations.

It is understood that additional antisense oligonucleotides can be constructed based on the TGF-β2 gene sequence.

In some embodiments, a TGF-β2-specific antisense oligonucleotide of this invention may have no more than one or two mismatches as compared to a target human TGF-β2.

In certain embodiments, a TGF-β2-specific antisense oligonucleotide of this invention may reduce a TGF-β2 transcript level by at least 60%, or at least 70%, or at least 80%, or at least 90%.

In additional embodiments, a TGF-β2-specific antisense oligonucleotide of this invention may be selective for TGF-β2 and reduce any TGF-β1 transcript level and any TGF-β3 transcript level by less than 10%, or less than 5%, or less than 1%.

In further embodiments, a therapeutically effective amount of an antisense agent for inhibiting or suppressing expression of TGF-β2 can be from 0.1 to 3000 mg per day, or 1 to 1000 mg per day, or 2 to 500 mg per day, or 2 to 200 mg per day.

In certain embodiments, a formulation of an antisense agent for inhibiting or suppressing expression of TGF-β2 can have a concentration of from 0.05 to 50 μM, or 0.1 to 25 μM, or 0.1 to 10 μM, or 0.1 to 7.5 μM, or 0.1 to 5 μM.

In certain embodiments, a method for using an antisense agent for inhibiting or suppressing expression of TGF-β2 can use a dosage of from 1 to 1000 mg/m²/day, or from 1 to 500 mg/m²/day, or from 1 to 250 mg/m²/day, or from 1 to 100 mg/m²/day, or from 1 to 50 mg/m²/day. Mean human body surface area can be about 1.6 to 1.9 m².

In additional embodiments, a method for using an antisense agent for inhibiting or suppressing expression of TGF-β2 can use a dosage of from 0.05 to 40 mg/kg/day, or from 0.1 to 30 mg/kg/day, or from 0.2 to 20 mg/m²/day, or from 0.3 to 10 mg/m²/day, or from 0.5 to 5 mg/m²/day. Mean human body weight can be about 60 kg.

In certain embodiments, agents of this disclosure for inhibiting or suppressing expression of TGF-β2 may be prepared from a lyophilized powder of the agent.

In some examples and embodiments, an agent may be a TGF-β2-specific antisense oligonucleotide selected from SEQ ID NOs: 1-136, and administered or used by injection or infusion at a dose of 4 μl/min at a dose level of 10 μM on the Days 1 to 7, or at a dose of 20 μM on Days 1 to 7, or at a dose of 40 μM on Days 1 to 7, or at a dose of 80 μM on Days 1 to 7. In some embodiments, an agent may be a TGF-2-specific antisense oligonucleotide selected from SEQ ID NOs: 9-136, and administered or used by injection or infusion at a dose of 4 μl/min at a dose level of 10 μM on the Days 1 to 7, or at a dose of 20 μM on Days 1 to 7, or at a dose of 40 μM on Days 1 to 7, or at a dose of 80 μM on Days 1 to 7.

In some examples and embodiments, an agent may be a TGF-β2 gene sequence-specific antisense oligonucleotide selected from SEQ ID NOs: 1-136, and administered or used by injection or infusion at a dose of 4 μl/min, or 2-8 μl/min, at a dose level of 2 μM on Days 1 to 7, or at a dose of 4 μM on Days 1 to 7, or at a dose of 8 μM on Days 1 to 7, or at a dose of 10 μM on Days 1 to 7. In some embodiments, an agent may be a TGF-β2 gene sequence-specific antisense oligonucleotide selected from SEQ ID NOs: 9-136, and administered or used by injection or infusion at a dose of 4 μl/min, or 2-8 μl/min, at a dose level of 2 μM on Days 1 to 7, or at a dose of 4 μM on Days 1 to 7, or at a dose of 8 μM on Days 1 to 7, or at a dose of 10 μM on Days 1 to 7.

Embodiments of this invention involving administration or use of a composition of an agent can ameliorate or suppress symptoms due to TGF-β2 induced proteins.

As used herein, the term agent can refer to a single active agent or a composition of a single active agent and a carrier.

Embodiments of this invention further include pharmaceutical compositions for inhibiting or suppressing expression of TGF-β2, or for treating or ameliorating the symptoms of cancer in a human or animal. The pharmaceutical compositions may contain a TGF-β2 inhibitor, artemisinin, pharmaceutically acceptable salts forms, esters, polymorphs or stereoisomers thereof, and any combination thereof, as well as a carrier. The TGF-β2 inhibitor may be selected from TGF-β2-specific antisense oligonucleotides such as SEQ ID NOs: 1-136, or SEQ ID NOs: 9-136, and chemically-modified variants thereof. The carrier may be sterile water for injection, saline, isotonic saline, or a combination thereof.

Importantly, a composition of this disclosure may be substantially free of excipients. Compositions of this invention which are substantially free of excipients have been found to be surprisingly stable in a carrier. In some embodiments, the composition may be stable for at least 14 days, or at least 21 days, or at least 28 days in a carrier at 37° C.

In additional embodiments, a pharmaceutical composition for infusion may contain less than 1% by weight of excipients, or less than 0.5% by weight of excipients, or less than 0.1% by weight of excipients.

Embodiments of this invention further contemplate therapeutic modalities in which a composition of this invention is administered or utilized in combination with a standard of care therapy for the disease. Examples of additional medicaments which may be administered or utilized in combination with a composition of this invention include anti-inflammatories, anti-inflammatory steroids, piperiquine, pyronaridine, curcumin, frankincense, Remdesivir, Sompraz D, Zifi CV/Zac D, CCM, Broclear, Budamate, Rapitus, Montek LC, low molecular weight heparine, prednisolone, Paracetamol, Vitamin B complex, Vitamin C, Pantoprozol, Doxycycline, Ivermectin, Zinc, Foracort Rotacaps inhalation, Injection Ceftriaxone, Tab Paracetamol, Injection Fragmin, Tablet Covifor, Azithromycin, Injection Dexamethasone, Injection Odndansetron, Tablet Multivitamin, Tablet Ascorbic Acid, Tablet Calcium Carbonate, and Tablet Zinc Sulfate.

Some TGF-β2-specific antisense oligonucleotide agents are given in U.S. Pat. Nos. 9,963,703, 9,758,786, and 8,476,246.

For example, the API trabedersen (OT-101) is a synthetic 18-mer S-ODN comprised of the bases adenine (A), thymine (T), guanine (G), and cytosine (C), with all 3′-5′ linkages modified to phosphorothioates. This sulfur modification makes the drug more resistant to degradation, resulting in an increased stability in vitro and in vivo. Its primary molecular structure, the nucleotide sequence, was designed to be complementary to a specific sequence of human transforming growth factor-beta 2 (TGF-β2) mRNA. This sequence and related sequences can be used for superior chemical and structural properties, biological activity, and specificity to achieve the best antisense effects in vitro and in vivo.

Chemical structure, exemplary of the phosphorothioate moieties (C-A-G), and the physical characteristics of trabedersen are shown in Table 2.

TABLE 2
Chemical and Physical Characteristics of Trabedersen

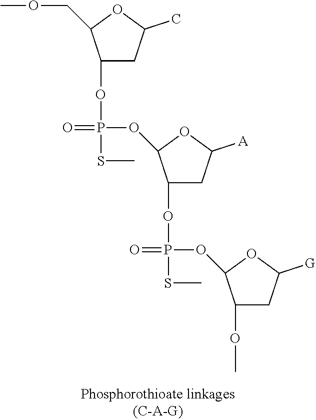

| Appearance: | White to off-white solid |
|---|---|
| Molecular Formula | C₁₇₇N₂₀₈N₆₀O₉₄P₁₇S₁₇Na₁₇ |
| salt, anhydrous): | |
| Molecular Weight | 6143 g/mol |
| (sodium salt, anhydrous): | |
| Number of moieties: | 18 |
| Backbone characteristics: | Phosphorothioates |
| Polymorphism: | No crystalline morphology in the solid |
| Water content: | Up to 15% |
| Solubility: | Very soluble in water (>50 mg/mL) |
| | Soluble in methanol (>8.5 mg/mL) |
| | Insoluble in iso-propanol |
| | (0.01 mg/mL) |
| pH of solution: | 6-8 |

The investigational medicinal product can be supplied as a sterile lyophilizate for solution for infusion in 50 mL glass vials (primary container) containing 7.37 mg trabedersen (intratumoral treatment) and in 20 R glass vials (primary container) containing 250 mg trabedersen (intravenous treatment), respectively. The finished drug product may contain no excipients. Glass vials may be used for parenterals. Sterile rubber stoppers appropriate for lyophilization can seal the glass vial. The stopper may be sealed with a crimping capsule that includes a colored flip-off cap. For clinical use, each vial can be provided within a white-colored folding box to protect the vials from light exposure and damage during transport. Both the glass vials and the folding boxes may be labeled according to local requirements. The primary as well as secondary containers of the closure system can fulfill international quality standards for the packaging of sterile solid drug products for injections. A kit can supply OT-101 as a lyophilized powder in 50-mL glass vials in different quantities, and specify total volume after dissolving (in mL) and resulting concentration (in μM).

This invention further provides kits comprising a lyophilized powder in a vial at a content of 250 mg each of one or more TGF-β2-specific antisense oligonucleotides selected from SEQ ID NOs: 1-136. The kit may contain the appropriate vial(s) and all necessary components of the application system, i.e., syringes, tube, and filter. OT-101 lyophilized powder can be dissolved in isotonic (0.9%) aqueous sodium chloride prior to use.

### Anti-Cancer Agents and Therapeutic Combinations

Embodiments of this invention include combinations of TGF-β2 inhibitors and anti-cancer agents such as irinotecan and irinotecan-containing formulations or regimens.

Examples of irinotecan-containing formulations, agents and/or regimens include a FOLFIRINOX agent, a NALIRIFOX agent, an IRIFOX agent, and an IRINOX agent. Dosages and times of administration of such irinotecan-containing formulations, agents and/or regimens are as known in the art.

An irinotecan-containing agent of this disclosure can be formulated in various forms, such as a small particle form, a micelle form, a hydrogel form, a liposome form, or an exosome form.

In certain embodiments, this invention provides therapeutic combinations of one or more antisense TGF-β2 inhibitors and an irinotecan-containing agent with nano-sized particles, nano-sized micelles, nano-sized hydrogels, nano-sized liposomes, or nano-sized exosomes.

Additional embodiments of this invention include therapeutic combinations of a TGF-β2 inhibitor and a FOLFIRINOX agent, a NALIRIFOX agent, an IRIFOX agent, or an IRINOX agent, along with standard-of-care therapies.

The unexpectedly advantageous synergistic effect of the therapeutic combination for cancer treatment of an antisense TGF-β2 inhibitor and an irinotecan-containing agent can be increased when patients are selected based on biomarkers for the clinical effects.

In treating cancer patients, use of a therapeutic combination of an irinotecan-containing agent in combination with an antisense TGF-β2 inhibitor can significantly increase patient overall survival. In some embodiments, therapeutic combination of an antisense TGF-β2 inhibitor and an irinotecan-containing agent can provide an unexpectedly advantageous synergistic effect based on clinical data.

### Synergy of Therapeutic Combinations

Embodiments of this invention can provide synergy for a therapeutic combination of an agent for inhibiting or suppressing expression of TGF-β2 with an agent containing a topoisomerase I inhibitor for treating or ameliorating the symptoms of cancer.

Examples of an irinotecan agent include irinotecan, irinotecan hydrochloride, irinotecan sucrose sulfate, and irinotecan sucrose sulfate salt forms.

Examples of topoisomerase I inhibitors include Camptothecin derivatives and analogues such as irinotecan, topotecan, rubitecan, and 9-aminocamptothecin.

In some embodiments, the anti-cancer use of the combination of an antisense agent for inhibiting or suppressing expression of TGF-β2 and an irinotecan-containing agent can be particularly effective for patients with high levels of certain biomarkers, for example TGF-β2 among others. Biomarkers can be used to select patients who will benefit from the combination therapy.

Examples of regimens using agents containing irinotecan include a FOLFIRINOX agent, a NALIRIFOX agent, an IRIFOX agent, and an IRINOX agent, as known in the art. In one example, NALIRIFOX is liposomal irinotecan formulated with oxaliplatin, 5-fluorouracil, and leucovorin. In one example, IRINOX is a combination of irinotecan formulated with oxaliplatin. In one example, FOLFIRINOX is a combination of irinotecan hydrochloride formulated with leucovorin calcium (folinic acid), fluorouracil, and oxaliplatin. In one example, FOLFIRI is a formulation of folinic acid (leucovorin, calcium folinate or FA), fluorouracil (5FU), and irinotecan. In one example, IRINOX is a formulation of irinotecan plus oxaliplatin.

A disadvantage of conventional therapies with irinotecan-containing agents alone is that not all cancer patients are suitable for such treatment. Irinotecan-containing agents can be used for cancer patients exhibiting low TGF-β2, which is a value showing low risk of progression. However, cancer patients who have high TGF-β2, which is a value showing risk of progression, and/or other biomarker values which suggest a risk for progression are typically treated only with palliative therapy such as gemcitabine.

Embodiments of this invention which combine an agent for inhibiting or suppressing expression of TGF-β2 with an agent containing irinotecan can exhibit the surprising advantage that overall survival and/or HRQOL can be improved for all cancer patients.

For example, embodiments of this invention utilizing a combination of a TGF-β2 inhibitor and an irinotecan-containing agent can be used for cancer patients who have low TGF-β2.

Further, embodiments of this invention can be used for cancer patients who have high TGF-β2 and/or other biomarker values which suggest a risk for progression.

Thus, embodiments of this invention which combine an antisense agent for inhibiting or suppressing expression of TGF-β2 with an agent containing irinotecan can be used advantageously for all cancer patients.

Agents containing irinotecan can be used against cancer, including pancreatic cancer, colorectal cancer, stomach cancer, cervical cancer, lung cancer, melanoma, skin cancer, breast cancer, prostate cancer, kidney cancer, ovarian cancer, thymus cancer, and multiple myeloma.

Embodiments of this invention can provide therapeutic combinations of an agent for inhibiting or suppressing expression of TGF-β2 with an agent containing a topoisomerase I inhibitor for treating or ameliorating the symptoms of cancer, including pancreatic cancer, colorectal cancer, stomach cancer, cervical cancer, lung cancer, melanoma, skin cancer, breast cancer, prostate cancer, kidney cancer, ovarian cancer, thymus cancer, and multiple myeloma. The topoisomerase I inhibitor can be irinotecan, irinotecan hydrochloride, irinotecan sucrose sulfate, irinotecan sucrose sulfate salt, a FOLFIRINOX agent, a NALIRIFOX agent, an IRIFOX agent, an IRINOX agent, topotecan, rubitecan, or 9-aminocamptothecin.

Numbered embodiments of this disclosure include the following:
(1) An antisense agent for inhibiting or suppressing expression of TGF-β2 in combination with an agent comprising irinotecan for use in treating or ameliorating the symptoms of cancer in a subject.
(2) Use of an antisense agent for inhibiting or suppressing expression of TGF-β2 in the preparation of a medicament for treating or ameliorating the symptoms of a cancer in a subject in combination with an agent comprising irinotecan.
(3) A method for treating or ameliorating the symptoms of cancer in a subject in need, the method comprising:
administering a therapeutically effective amount of an antisense agent for inhibiting or suppressing expression of TGF-β2 to the subject; and
administering a therapeutically effective amount of an agent comprising irinotecan to the subject.
(4) The agent, use or method of any of embodiments 1-3, wherein the agent comprising irinotecan is a FOLFIRINOX agent, a NALIRIFOX agent, an IRIFOX agent, or an IRINOX agent.
(5) The agent, use or method of any of embodiments 1-4, wherein the agent comprising irinotecan comprises a nano-sized particle, a nano-sized micelle, a nano-sized liposome, or a nano-sized exosome.
(6) The agent, use or method of any of embodiments 1-5, wherein the agent for inhibiting or suppressing expression of TGF-β2 and the agent comprising irinotecan are administered concurrently, simultaneously, sequentially, or separately in time.
(7) The agent, use or method of any of embodiments 1-6, wherein the agent for inhibiting or suppressing expression of TGF-β2 and the agent comprising irinotecan are administered separately or in combination by injection or infusion.
(8) The agent, use or method of any of embodiments 1-7, wherein the cancer is a pancreatic cancer, a colorectal cancer, a stomach cancer, a cervical cancer, a lung cancer, melanoma, a skin cancer, a breast cancer, a prostate cancer, a kidney cancer, an ovarian cancer, a thymus cancer, and multiple myeloma.
(9) The agent, use or method of any of embodiments 1-8, wherein the agent for inhibiting or suppressing expression of TGF-β2 is a TGF-β2-specific antisense oligonucleotide complementary to a TGF-β2 transcript and 15-30 nucleotides in length.
(10) The agent, use or method of any of embodiments 1-9, wherein the agent for inhibiting or suppressing expression of TGF-β2 is a TGF-β2-specific antisense oligonucleotides complementary to a TGF-β2 pre-RNA, pre-mRNA or mRNA and 18-21 nucleotides in length.
(11) The agent, use or method of any of embodiments 1-10, wherein the agent for inhibiting or suppressing expression of TGF-β2 is one or more TGF-β2-specific antisense oligonucleotides complementary to a TGF-β2 transcript in Table 1 and chemically-modified variants thereof, LNA variants thereof, gapmer variants thereof, and any combination or pooling thereof.
(12) The agent, use or method of any of embodiments 1-11, wherein the TGF-β2-specific antisense oligonucleotides have no more than one or two mismatches as compared to a target human TGF-β2.
(13) The agent, use or method of any of embodiments 1-12, wherein the TGF-β2-specific antisense oligonucleotides reduce a TGF-β2 transcript level by at least 60%, or at least 70%, or at least 80%, or at least 90%.
(14) The agent, use or method of any of embodiments 1-13, wherein the TGF-β2-specific antisense oligonucleotides reduce any TGF-β1 transcript level and any TGF-β3 transcript level by less than 10%, or less than 5%, or less than 1%.
(15) The agent, use or method of any of embodiments 1-14, wherein the TGF-β2-specific antisense oligonucleotides have one or more nucleotides chemically modified as a phosphorothioate internucleoside linkage, a methoxypropylphosphonate internucleoside linkage, an aminophosphoro linkage to a morpholino group, a 2′-OMe ribose group, a 2′-MOE methoxyethyl ribose group, a 2′-4′ constrained methoxyethyl bicyclic ribose group, a 2′-4′ constrained ethyl bicyclic ribose group, an LNA ribose group, a 2′-F ribose group, or a 5-methylcytodine base.
(16) The agent, use or method of any of embodiments 1-15, wherein the antisense agent is conjugated to a polyethylene glycol, a lipid, or a triantenarry N-acteyl-galactosamine.
(17) The agent, use or method of any of embodiments 1-16, wherein the agent for inhibiting or suppressing expression of TGF-β2 comprises a carrier of sterile water for injection, saline, isotonic saline, or a combination thereof, which may be the same or different for each agent.
(18) The agent, use or method of any of embodiments 1-17, wherein the agent for inhibiting or suppressing expression of TGF-β2 is substantially free of excipients.
(19) The agent, use or method of any of embodiments 1-18, wherein the agent for inhibiting or suppressing expression of TGF-β2 is stable in a carrier substantially free of excipients for at least 14 days at 37° C.
(20) The agent, use or method of any of embodiments 1-19, wherein patients are selected who have previously been treated with any agent comprising irinotecan.
(21) The agent, use or method of any of embodiments 1-20, comprising using one or more biomarkers to select subjects who benefit from the agent, use or method.
(22) The agent, use or method of any of embodiments 1-21, wherein patients are selected who have an elevated level of TGF-β2.
(23) The agent, use or method of any of embodiments 1-22, wherein the one or more biomarkers are a level of a tumor mutation burden (TMB), a level of a tumor neoantigen, a level of clinical hypoxia, or a combination thereof.
(24) The agent, use or method of any of embodiments 1-23, wherein the one or more biomarkers are a level of a tumor mutation burden, a level of a reduced neoantigen load determined in a tumor microenvironment, a level of a reduced macrophage determined in a tumor microenvironment, a level of a reduced mesenchymal stem cell MSC determined in a tumor microenvironment, a level of a type 2 T-helper cell Th2 determined in a tumor microenvironment, and a combination thereof.
(25) The method, agent or use of any of embodiments 1-24, wherein the subject upon the administration or use has an improved level of at least one of the one or more biomarkers as compared to a level found in a healthy patient.
(26) The agent, use or method of any of embodiments 1-25, wherein the administration or use decreases mortality rate at month 6, 12, 18, 24, 30, or 36.
(27) The agent, use or method of any of embodiments 1-26, wherein the administration or use increases survival rate at month 6, 12, 18, 24, 30, or 36.
(28) The agent, use or method of any of embodiments 1-27, wherein the administration or use increases health-related quality of life (HRQOL) by 15%, or 25%, or 35%, or 45%.
(29) The agent, use or method of any of embodiments 1-28, in combination with any one or more medicaments comprising a targeted cancer drug, a cancer growth blocker, an EGFR inhibitor, and combinations thereof.
(30) The agent, use or method of any of embodiments 1-29, in combination with any one or more medicaments selected from bevacizumab, everolimus, belzutifan, dabrafenib, trametinib, and combinations thereof.
(31) The agent, use or method of any of embodiments 1-30, in combination with any one or more medicaments which are cancer growth blockers selected from an angiogenesis inhibitor, a histone deacetylase inhibitor, a hedgehog blocker, an mTOR inhibitor, a p53 inhibitor, a PARP inhibitor, a proteasome inhibitor, a tyrosine kinase inhibitor, and combinations thereof.
(32) The agent, use or method of any of embodiments 1-31, in combination with any one or more medicaments which are EGFR inhibitors selected from erlotinib, gefitinib, afatinib, osimertinib, dacomitininb, and combinations thereof.
(33) The agent, use or method of any of embodiments 1-32, in combination with a standard of care treatment for cancer.
(34) The agent, use or method of any of embodiments 1-33, in combination with radiation therapy or electric field therapy.
(35) A kit for treating or ameliorating the symptoms of cancer, the kit comprising:
a therapeutically effective amount of an antisense agent for inhibiting or suppressing expression of TGF-β2; and
a therapeutically effective amount of an agent comprising irinotecan.
(36) The kit of embodiment 35, wherein the cancer is a pancreatic cancer, a colorectal cancer, a stomach cancer, a cervical cancer, a lung cancer, melanoma, a skin cancer, a breast cancer, a prostate cancer, a kidney cancer, an ovarian cancer, a thymus cancer, and multiple myeloma.
(37) The kit of any of embodiments 35-36, wherein the agent comprising irinotecan is a FOLFIRINOX agent, a NALIRIFOX agent, an IRIFOX agent, or an IRINOX agent.
(38) The kit of any of embodiments 35-37, wherein the agent comprising irinotecan comprises a nano-sized particle, a nano-sized micelle, a nano-sized liposome, or a nano-sized exosome.
(39) The kit of any of embodiments 35-38, wherein the agent for inhibiting or suppressing expression of TGF-β2 and the agent comprising irinotecan are administered concurrently, simultaneously, sequentially, or separately in time.
(40) The kit of any of embodiments 35-39, wherein the agent for inhibiting or suppressing expression of TGF-β2 and the agent comprising irinotecan are administered separately or in combination by injection or infusion.
(41) The kit of any of embodiments 35-40, wherein the agent for inhibiting or suppressing expression of TGF-β2 is a TGF-β2-specific antisense oligonucleotide complementary to a TGF-β2 transcript and 15-30 nucleotides in length.
(42) The kit of any of embodiments 35-41, wherein the agent for inhibiting or suppressing expression of TGF-β2 is a TGF-β2-specific antisense oligonucleotides complementary to a TGF-β2 pre-RNA, pre-mRNA or mRNA and 18-21 nucleotides in length.
(43) The kit of any of embodiments 35-42, wherein the agent for inhibiting or suppressing expression of TGF-β2 is one or more TGF-β2-specific antisense oligonucleotides complementary to a TGF-β2 transcript as given in Table 1.
(44) The kit of any of embodiments 35-43, wherein the TGF-β2-specific antisense oligonucleotides have no more than one or two mismatches as compared to a target human TGF-β2.
(45) The kit of any of embodiments 35-44, wherein the TGF-β2-specific antisense oligonucleotides reduce a TGF-β2 transcript level by at least 60%, or at least 70%, or at least 80%, or at least 90%.
(46) The kit of any of embodiments 35-45, wherein the TGF-β2-specific antisense oligonucleotides reduce any TGF-β1 transcript level and any TGF-β3 transcript level by less than 10%, or less than 5%, or less than 1%.
(47) The kit of any of embodiments 35-46, wherein the TGF-β2-specific antisense oligonucleotides have one or more nucleotides chemically modified as a phosphorothioate internucleoside linkage, a methoxypropylphosphonate internucleoside linkage, an aminophosphoro linkage to a morpholino group, a 2′-OMe ribose group, a 2′-MOE methoxyethyl ribose group, a 2′-4′ constrained methoxyethyl bicyclic ribose group, a 2′-4′ constrained ethyl bicyclic ribose group, an LNA ribose group, a 2′-F ribose group, or a 5-methylcytodine base.
(48) The kit of any of embodiments 35-47, wherein the antisense agent is conjugated to a polyethylene glycol, a lipid, or a triantenarry N-acteyl-galactosamine.
(49) The kit of any of embodiments 35-48, wherein the agent for inhibiting or suppressing expression of TGF-β2 comprises a carrier of sterile water for injection, saline, isotonic saline, or a combination thereof, which may be the same or different for each agent.
(50) The kit of any of embodiments 35-49, wherein the agent for inhibiting or suppressing expression of TGF-β2 is substantially free of excipients.
(51) The kit of any of embodiments 35-50, wherein the agent for inhibiting or suppressing expression of TGF-β2 is stable in a carrier substantially free of excipients for at least 14 days at 37° C.

All publications including patents, patent application publications, and non-patent publications referred to in this description, as well as the sequence listing are each expressly incorporated herein by reference in their entirety for all purposes.

Although the foregoing disclosure has been described in detail by way of example for purposes of clarity of understanding, it will be apparent to the artisan that certain changes and modifications are comprehended by the disclosure and may be practiced without undue experimentation within the scope of the appended claims, which are presented by way of illustration not limitation. This invention includes all such additional embodiments, equivalents, and modifications. This invention includes any combinations or mixtures of the features, materials, elements, or limitations of the various illustrative components, examples, and claimed embodiments.

It is emphasized herein according to common practice the features of the drawings have arbitrary scale and are intended to cover similar features that may be arbitrarily expanded or reduced.

### EXAMPLES

Example 1. Methods and agents of this disclosure for suppressing TGF-β2 can be used against pancreatic cancer.

A study of clinical results for pancreatic cancer patients was performed which showed improvement in overall survival (OS) with reduced TGF-β2 expression. This result was surprising because neither TGF-β1 nor TGF-β3 isoforms were found to be significantly linked to increased survival in pancreatic cancer. Thus, this clinical study confirmed TGF-β2 alone as a therapeutic target in pancreatic cancer.

Kaplan-Meier Plotter was used to calculate patient survival to determine the connection between improved overall survival in pancreatic cancer and reduced TGF-β2 expression.

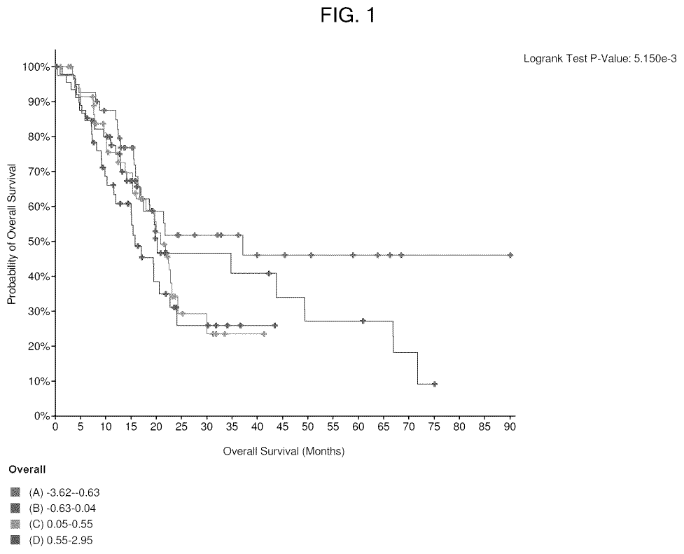

Results of the study for pancreatic cancer are shown in FIG. 1. FIG. 1 shows highly significant (logrank P=0.00515) improvement in overall survival for pancreatic cancer patients with reduced TGF-β2. As understood in the art, logrank p-values below about 0.05 (or <0.1) confirm that the differences are significant. The improvement found in overall survival with reduced TGF-β2 expression was a significant increase from 16 months for the high TGF-β2 expression cohort to 37 months for the low TGF-β2 expression cohort.

The study of FIG. 1 used Quartiles of TGFB2 expression from Z-scores of mRNA expression (log 2 RSEM-UQ). Quartile statistics are shown in Table 3. Overall and other survival calculations are shown in Table 4.

TABLE 3
TGF-β2 Quartile statistics for pancreatic cancer

| | Number of | Number of | Median Months Overall | |
|---|---|---|---|---|
| Quartile | Cases | Events | (95% CI) | |
| (A) −3.62-−0.63 | 82 | 35 | 37.12 | (17.48-NA) |
| (B) −0.63-0.04 | 96 | 49 | 20.17 | (18.67-49.34) |
| (C) 0.05-0.55 | 87 | 48 | 20.84 | (17.92-23.08) |
| (D) 0.55-2.95 | 90 | 53 | 15.81 | (15.05-20.61) |

TABLE 4
Overall and other survival calculations
for TGF-β2 in pancreatic cancer

| Survival Type | Number of Patients | p-Value | q-Value |
|---|---|---|---|
| Disease Free | 207 | 2.016e−4 | 8.063e−4 |
| Overall | 355 | 5.150e−3 | 0.0103 |
| Progression Free | 177 | 0.0155 | 0.0207 |

Example 2. Methods and agents of this disclosure for suppressing TGF-β2 can be used against pancreatic cancer.

A study of clinical results for pancreatic cancer patients was performed which showed improvement in overall survival (OS) with reduced TGF-β2 expression. Additional inclusion criterion: For this study, patients with no post-operative follow up cancer therapy were selected.

Kaplan-Meier Plotter was used to calculate patient survival to determine the connection between improved overall survival in pancreatic cancer and reduced TGF-β2 expression.

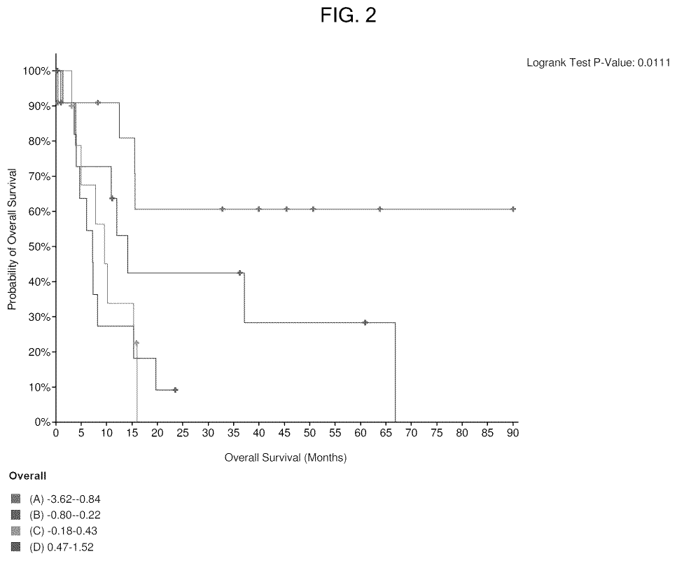

Results of the study for pancreatic cancer are shown in FIG. 2. FIG. 2 shows highly significant (logrank P=0.0111) improvement in overall survival for pancreatic cancer patients with reduced TGF-β2. The improvement found in overall survival with reduced TGF-β2 expression was a significant increase from 7 months for the high TGF-β2 expression cohort to at least 14 months for a low TGF-β2 expression cohort (B).

The study of FIG. 2 used Quartiles of TGFB2 from mRNA expression z-scores relative to all samples (log RNA Seq V2 RSEM). Quartile statistics are shown in Table 5. Overall and other survival calculations are shown in Table 6.

TABLE 5
TGF-β2 Quartile statistics for pancreatic cancer

| | Number of | Number of | Median Months Overall |
|---|---|---|---|
| Quartile | Cases | Events | (95% CI) |
| (A) −3.62-−0.84 | 11 | 4 | NA |
| (B) −0.80-−0.22 | 11 | 8 | 14.13 (10.97-NA) |
| (C) −0.18-0.43 | 11 | 8 | 9.59 (5.03-NA) |
| (D) 0.47-1.52 | 12 | 10 | 7.19 (4.73-NA) |

TABLE 6
Overall and other survival calculations
for TGF-β2 agents in pancreatic cancer

| Survival Type | Number of Patients | p-Value | q-Value |
|---|---|---|---|
| Disease Free | 31 | 0.0105 | 0.0111 |
| Overall | 45 | 0.0111 | 0.0111 |

Example 3. A study of clinical results for pancreatic cancer patients was performed which showed improvement in overall survival (OS) with reduced hypoxia. Thus, patients can be selected based on hypoxia as a biomarker.

Kaplan-Meier Plotter was used to calculate patient survival to determine the connection between improved overall survival in pancreatic cancer and reduced hypoxia.

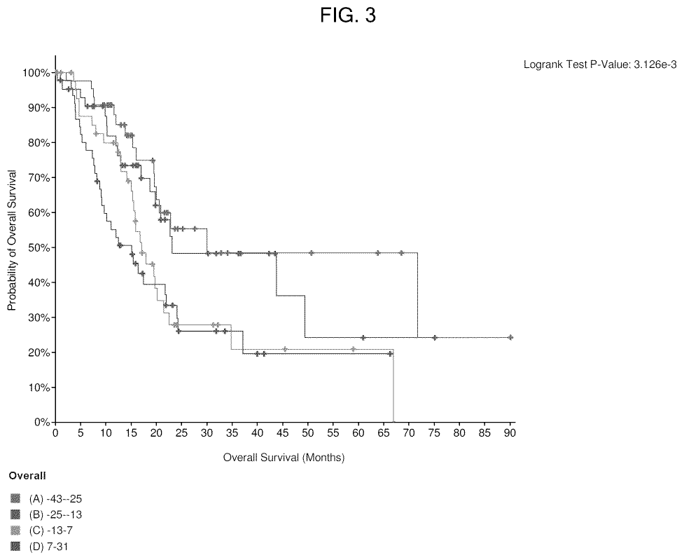

Results of the study for pancreatic cancer are shown in FIG. 3. FIG. 3 shows highly significant (logrank P=0.003126) improvement in overall survival for pancreatic cancer patients with reduced hypoxia. The improvement found in overall survival with reduced hypoxia was a significant increase from 15 months for the high hypoxia cohort to 30 months for a low hypoxia cohort.

The study of FIG. 3 used Quartiles of Buffa Hypoxia Score as biomarker for selecting patients. Quartile statistics are shown in Table 7. Overall and other survival calculations are shown in Table 8.

TABLE 7
Hypoxia Quartile statistics for pancreatic cancer

| | Number of | Number of | Median Months Overall | |
|---|---|---|---|---|
| Quartile | Cases | Events | (95% CI) | |
| (A) −43-−25 | 44 | 16 | 30.02 | (19.96-NA) |
| (B) −25-−13 | 44 | 18 | 23.08 | (19.82-NA) |
| (C) −13-7 | 44 | 27 | 17.03 | (15.32-22.49) |
| (D) 7-31 | 45 | 31 | 15.12 | (9.14-24.07) |

TABLE 8
Overall and other survival calculations in pancreatic cancer

| Survival Type | Number of Patients | p-Value | q-Value |
|---|---|---|---|
| Progression Free | 177 | 5.466e−4 | 2.186e−3 |
| Disease-specific | 171 | 2.896e−3 | 4.168e−3 |
| Overall | 177 | 3.126e−3 | 4.168e−3 |

Example 4. A study of clinical results for pancreatic cancer patients was performed which showed improvement in overall survival (OS) with reduced tumor mutation burden (TMB). Thus, patients can be selected based on tumor mutation burden (TMB) as a biomarker.

Kaplan-Meier Plotter was used to calculate patient survival to determine the connection between improved overall survival in pancreatic cancer and reduced TMB.

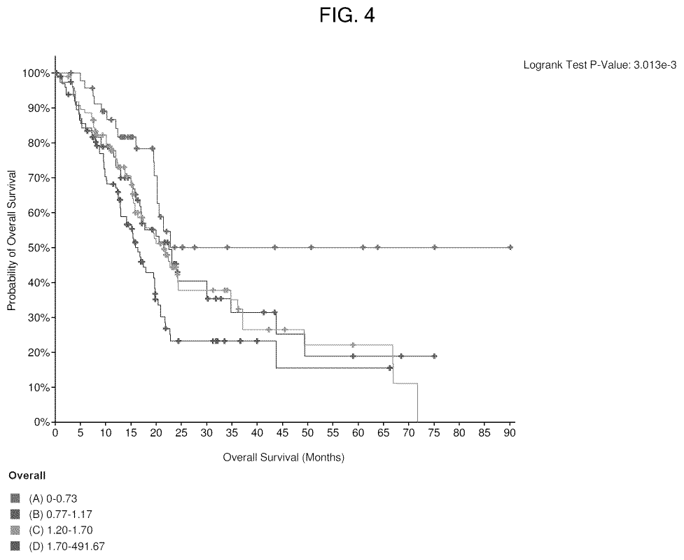

Results of the study for pancreatic cancer are shown in FIG. 4. FIG. 4 shows highly significant (logrank P=0.003013) improvement in overall survival for pancreatic cancer patients with reduced TMB. The improvement found in overall survival with reduced TMB was a significant increase from 16 months for the high TMB cohort to at least 22 months for a low TMB cohort (B).

The study of FIG. 4 used Quartiles of TMB (nonsynonymous) as biomarker for selecting patients. Quartile statistics are shown in Table 9. Overall and other survival calculations are shown in Table 10.

TABLE 9
TMB Quartile statistics for pancreatic cancer

| | Number of | Number of | Median Months Overall |
|---|---|---|---|
| Quartile | Cases | Events | (95% CI) |
| (A) 0-0.73 | 49 | 16 | NA |
| (B) 0.77-1.17 | 79 | 43 | 22.49 (17.00-34.82) |
| (C) 1.20-1.70 | 101 | 57 | 21.42 (16.37-34.79) |
| (D) 1.70-491.67 | 100 | 63 | 16.36 (12.95-19.81) |

TABLE 10
Overall and other survival calculations in pancreatic cancer

| Survival Type | Number of Patients | p-Value | q-Value |
|---|---|---|---|
| Overall | 329 | 3.013e−3 | 0.0121 |
| Disease Free | 182 | 0.103 | 0.153 |

Example 5. Methods and agents of this disclosure for suppressing TGF-β2 can be used against pancreatic cancer in combination with treatment with irinotecan agents.

A study of clinical results for pancreatic cancer patients was performed which showed improvement in overall survival (OS) with reduced TGF-β2 expression where patients were selected based on treatment with irinotecan. This result was surprising because improvement in overall survival (OS) with reduced TGF-β2 expression was not observed where patients were also treated with gemcitabine alone, fluorouracil alone, oxaliplatin alone, or leucovorin alone.

Kaplan-Meier Plotter was used to calculate patient survival to determine the connection between improved overall survival in pancreatic cancer and reduced TGF-β2 expression when patients were also treated with irinotecan.

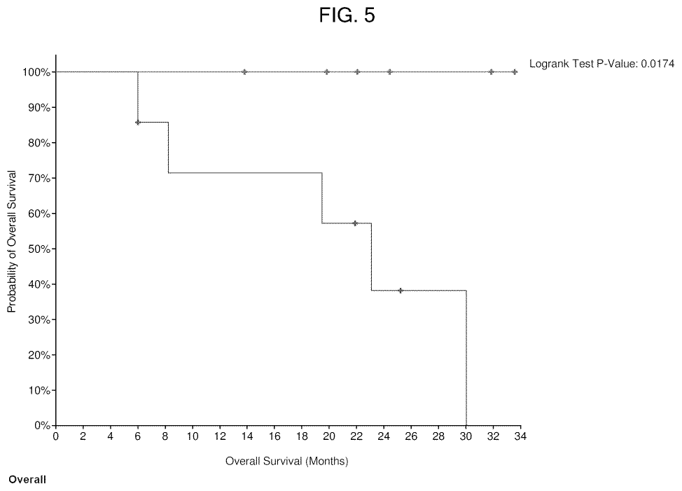

Results of the study for pancreatic cancer are shown in FIG. 5. FIG. 5 shows highly significant (logrank P=0.0174) improvement in overall survival for pancreatic cancer patients with reduced TGF-β2. The improvement found in overall survival with reduced TGF-β2 expression was a significant increase from 23 months for the high TGF-β2 expression cohort to much longer than 34 months for the low TGF-β2 expression cohort. The significant lengthening of overall survival indicates among other things that drug resistance effects are being avoided.

These results in FIG. 5 show the surprising synergy for increasing overall survival in the treatment of pancreatic cancer using therapy that combines reducing TGF-β2 expression with an irinotecan-containing agent. These results were especially surprising because none of oxaliplatin, leucovorin, and fluorouracil showed significant synergy in combination with reduced TGF-β2 expression.

The study of FIG. 5 used Quartiles of TGFB2 from mRNA expression z-scores relative to all samples (log RNA Seq V2 RSEM). Quartile statistics are shown in Table 11. Overall and other survival calculations are shown in Table 12.

TABLE 11
TGF-β2 Quartile statistics for pancreatic cancer

| | Number of | Number of | Median Months Overall |
|---|---|---|---|
| Quartile | Cases | Events | (95% CI) |
| (A) −1.75-0.17 | 6 | 0 | >>34 |
| (B) 0.33-2.64 | 7 | 5 | 23.08 (8.22-NA) |

TABLE 12
Overall and other survival calculations
for TGF-β2 in pancreatic cancer

| Survival Type | Number of Patients | p-Value | q-Value |
|---|---|---|---|
| Overall | 13 | 0.0174 | 0.0422 |
| Disease-specific | 12 | 0.0211 | 0.0422 |

Example 6. Methods and agents of this disclosure for suppressing TGF-β2 can be used against pancreatic cancer in combination with treatment with irinotecan agents.

A study of clinical results for pancreatic cancer patients was performed which showed improvement in overall survival (OS) with reduced TGF-β2 expression where patients were selected based on treatment with irinotecan.

Kaplan-Meier Plotter was used to calculate patient survival to determine the connection between improved overall survival in pancreatic cancer and reduced TGF-β2 expression when patients were also treated with irinotecan.

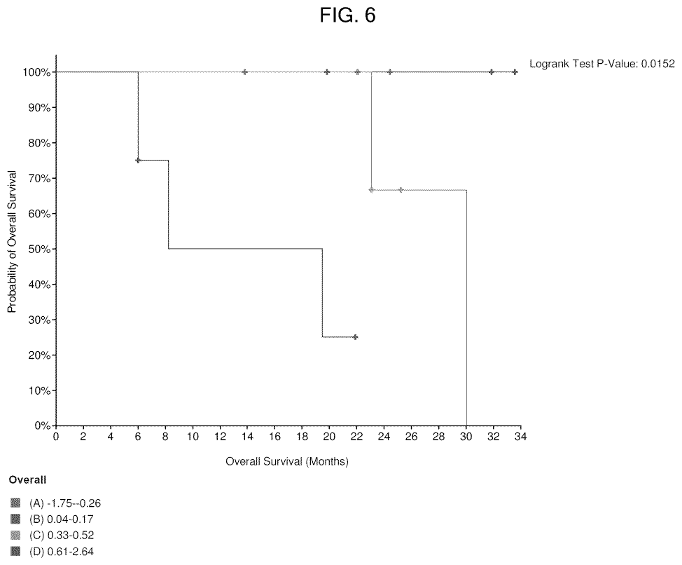

Results of the study for pancreatic cancer are shown in FIG. 6. FIG. 6 shows highly significant (logrank P=0.0152) improvement in overall survival for pancreatic cancer patients with reduced TGF-β2. The improvement found in overall survival with reduced TGF-β2 expression was a significant increase from 8 months for the high TGF-β2 expression cohort to at least 30 months for a lower TGF-β2 expression cohort (C). The significant lengthening of overall survival indicates among other things that drug resistance effects are being avoided.

The study of FIG. 6 used Quartiles of TGFB2 from mRNA expression z-scores relative to all samples (log RNA Seq V2 RSEM). Quartile statistics are shown in Table 13. Overall survival calculations are shown in Table 14.

TABLE 13
TGF-β2 Quartile statistics for pancreatic cancer

| | Number of | Number of | Median Months Overall |
|---|---|---|---|
| Quartile | Cases | Events | (95% CI) |
| (A) −1.75-−0.26 | 3 | 0 | NA |
| (B) 0.04-0.17 | 3 | 0 | NA |
| (C) 0.33-0.52 | 3 | 2 | 30.02 (23.08-NA) |
| (D) 0.61-2.64 | 4 | 3 | 8.22 (6.02-NA) |

TABLE 14
Overall survival calculations for TGF-β2 in pancreatic cancer

| Survival Type | Number of Patients | p-Value | q-Value |
|---|---|---|---|
| Overall | 13 | 0.0152 | 0.0606 |

Example 7. Methods and agents of this disclosure for suppressing TGF-β2 can be used against pancreatic cancer in combination with treatment with irinotecan agents.

A study of clinical results for pancreatic cancer patients was performed which showed improvement in overall survival (OS) with reduced TGF-β2 expression where patients were selected based on treatment with irinotecan-containing FOLFIRINOX agents.

Kaplan-Meier Plotter was used to calculate patient survival to determine the connection between improved overall survival in pancreatic cancer and reduced TGF-β2 expression when patients were also treated with irinotecan-containing FOLFIRINOX agents.

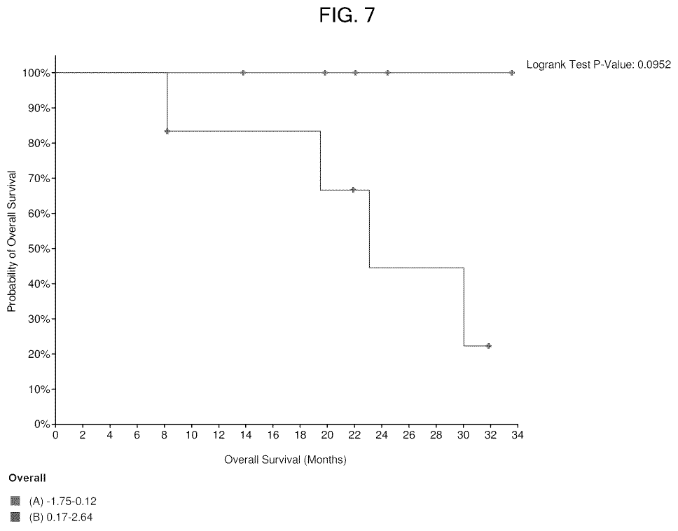

Results of the study for pancreatic cancer are shown in FIG. 7. FIG. 7 shows significant (logrank P=0.0952) improvement in overall survival for pancreatic cancer patients with reduced TGF-2. The improvement found in overall survival with reduced TGF-β2 expression was a significant increase from 23 months for the high TGF-β2 expression cohort to at least 34 months for a lower TGF-β2 expression cohort (B). The significant lengthening of overall survival indicates among other things that drug resistance effects are being avoided.

The study of FIG. 7 used Quartiles of TGF-β2 from mRNA expression z-scores relative to all samples (log RNA Seq V2 RSEM). Quartile statistics are shown in Table 15. Overall survival calculations are shown in Table 16.

TABLE 15
TGF-β2 Quartile statistics for pancreatic cancer

| | Number of | Number of | Median Months Overall |
|---|---|---|---|
| Quartile | Cases | Events | (95% CI) |
| (A) −1.75-0.12 | 5 | 0 | NA |
| (B) 0.17-2.64 | 6 | 4 | 23.08 (19.46-NA) |

TABLE 16
Overall survival calculations for TGF-β2 in pancreatic cancer

| Survival Type | Number of Patients | p-Value | q-Value |
|---|---|---|---|
| Overall | 11 | 0.0952 | 0.175 |
| Disease-specific | 10 | 0.117 | 0.175 |

Example 8. Methods and agents of this disclosure for suppressing TGF-β2 can be used against pancreatic cancer where biomarkers can be used to select patients who benefit from the agents.

A study of clinical results for pancreatic cancer patients was performed which showed improvement in overall survival (OS) with reduced TGF-β2 expression where biomarkers were used to select patients who benefited from the agents. Biomarkers of significance against pancreatic cancer were reduced tumor mutation burden, reduced neoantigen load determined in a tumor microenvironment, reduced macrophage determined in a tumor microenvironment, reduced mesenchymal stem cells (MSC) determined in a tumor microenvironment, and increased type 2 T-helper cells (Th2) determined in a tumor microenvironment. Any combination of these biomarkers can also be used.

Kaplan-Meier Plotter was used to calculate patient survival to determine the connection between improved overall survival in pancreatic cancer and reduced TGF-β2 expression where biomarkers were used to select patients who benefited from the agents.

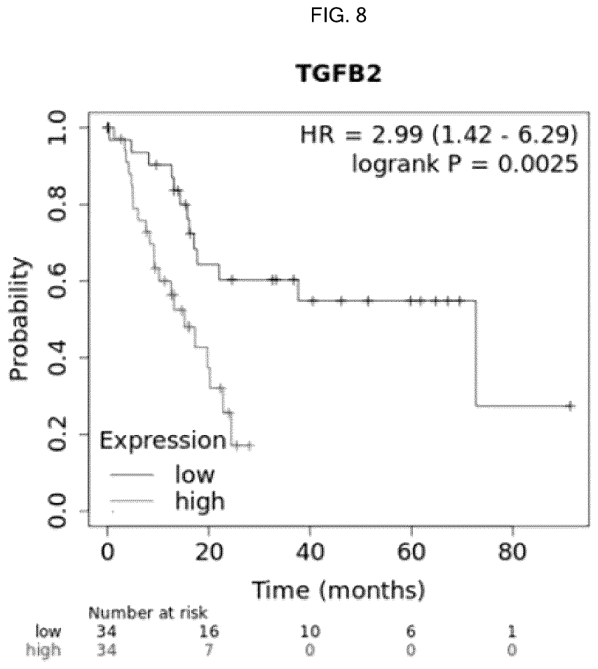

Results of the study for pancreatic cancer are shown in FIG. 8. FIG. 8 shows significant (logrank P=0.0025) improvement in overall survival for pancreatic cancer patients with reduced TGF-β2 where reduced macrophage determined in a tumor microenvironment was used to select patients. The improvement found in overall survival with reduced TGF-β2 expression and reduced macrophage was a significant increase from 15 months for the high TGF-β2 expression cohort to 73 months for a low TGF-β2 expression cohort. The significant lengthening of overall survival indicates among other things that drug resistance effects are being avoided.

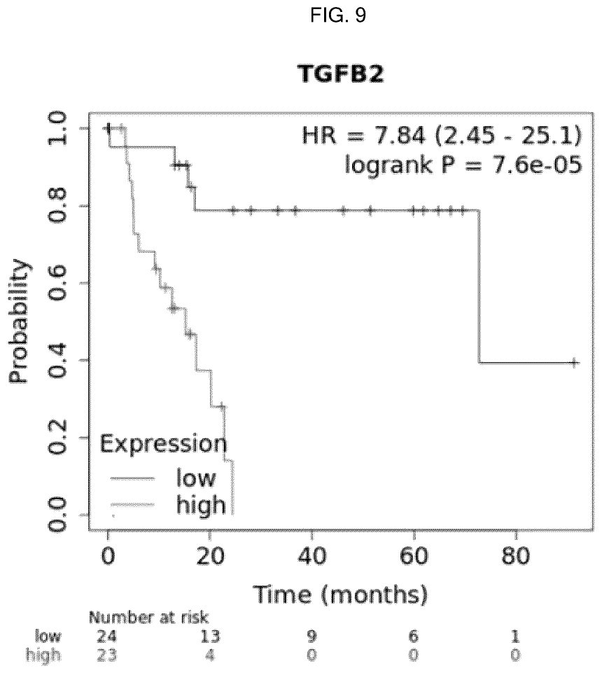

Similar results were obtained for additional biomarkers with p-values as follows:
reduced tumor mutation burden (logrank P=0.079),
reduced neoantigen load determined in a tumor microenvironment (logrank P=0.051),
reduced mesenchymal stem cells (MSC) determined in a tumor microenvironment (logrank P=0.031),
increased type 2 T-helper cells (Th2) determined in a tumor microenvironment, (logrank P=0.025),
a combination of reduced tumor mutation burden and reduced MO macrophage (logrank P=0.0018), and
a combination of reduced neoantigen load determined in a tumor microenvironment and reduced MO macrophage (see FIG. 9, logrank P=0.000076).

What is claimed is:

1. An antisense agent for inhibiting or suppressing expression of TGF-β2 in combination with an agent comprising irinotecan for use in treating or ameliorating the symptoms of cancer in a subject.

2. Use of an antisense agent for inhibiting or suppressing expression of TGF-β2 in the preparation of a medicament for treating or ameliorating the symptoms of a cancer in a subject in combination with an agent comprising irinotecan.

3. A method for treating or ameliorating the symptoms of cancer in a subject in need, the method comprising:
administering a therapeutically effective amount of an antisense agent for inhibiting or suppressing expression of TGF-β2 to the subject; and
administering a therapeutically effective amount of an agent comprising irinotecan to the subject.

4. The agent, use or method of any of claims 1-3, wherein the agent comprising irinotecan is a FOLFIRINOX agent, a NALIRIFOX agent, an IRIFOX agent, or an IRINOX agent.

5. The agent, use or method of any of claims 1-4, wherein the agent comprising irinotecan comprises a nano-sized particle, a nano-sized micelle, a nano-sized liposome, or a nano-sized exosome.

6. The agent, use or method of any of claims 1-5, wherein the agent for inhibiting or suppressing expression of TGF-β2 and the agent comprising irinotecan are administered concurrently, simultaneously, sequentially, or separately in time.

7. The agent, use or method of any of claims 1-6, wherein the agent for inhibiting or suppressing expression of TGF-β2 and the agent comprising irinotecan are administered separately or in combination by injection or infusion.

8. The agent, use or method of any of claims 1-7, wherein the cancer is a pancreatic cancer, a colorectal cancer, a stomach cancer, a cervical cancer, a lung cancer, melanoma, a skin cancer, a breast cancer, a prostate cancer, a kidney cancer, an ovarian cancer, a thymus cancer, and multiple myeloma.

9. The agent, use or method of any of claims 1-8, wherein the agent for inhibiting or suppressing expression of TGF-β2 is a TGF-β2-specific antisense oligonucleotide complementary to a TGF-β2 transcript and 15-30 nucleotides in length.

10. The agent, use or method of any of claims 1-9, wherein the agent for inhibiting or suppressing expression of TGF-β2 is a TGF-β2-specific antisense oligonucleotides complementary to a TGF-β2 pre-RNA, pre-mRNA or mRNA and 18-21 nucleotides in length.

11. The agent, use or method of any of claims 1-10, wherein the agent for inhibiting or suppressing expression of TGF-β2 is one or more TGF-β2-specific antisense oligonucleotides complementary to a TGF-β2 transcript as follows (Table 1):

| SEQ ID NO: | Ref. | ANTISENSE SEQUENCE |
|---|---|---|
| 1 | 1369-2613 | CGGCATGTCT ATTTTGTA |
| 2 | — | GTTCGTTTAG AGAACAGATC |
| 3 | — | TAAAGTTCGT TTAGAGAACA G |
| 4 | — | AGCCCTGTAT ACGAC |
| 5 | — | GTAGGTAAAA ACCTAATAT |
| 6 | — | CGTTTAGAGA ACAGATCTAC |
| 7 | — | CATTGTAGAT GTCAAAAGCC |
| 8 | — | CTCCCTCATG GTGGCAGTTG A |
| 9 | 178-195 | TTTGTTCCTG GATGACTC |
| 10 | 179-196 | GTTTGTTCCT GGATGACT |
| 11 | 180-197 | AGTTTGTTCC TGGATGAC |
| 12 | 181-198 | CAGTTTGTTC CTGGATGA |
| 13 | 182-199 | TCAGTTTGTT CCTGGATG |
| 14 | 183-200 | CTCAGTTTGT TCCTGGAT |
| 15 | 716-733 | TGTGTGTGTG TGCGTGTG |
| 16 | 717-734 | GTGTGTGTGT GTGCGTGT |
| 17 | 718-735 | TGTGTGTGTG TGTGCGTG |
| 18 | 719-736 | GTGTGTGTGT GTGTGCGT |
| 19 | 720-737 | TGTGTGTGTG TGTGTGCG |
| 20 | 721-738 | GTGTGTGTGT GTGTGTGC |
| 21 | 722-739 | TGTGTGTGTG TGTGTGTG |
| 22 | 723-740 | GTGTGTGTGT GTGTGTGT |
| 23 | 724-741 | TGTGTGTGTG TGTGTGTG |
| 24 | 725-742 | GTGTGTGTGT GTGTGTGT |
| 25 | 726-743 | TGTGTGTGTG TGTGTGTG |
| 26 | 727-744 | GTGTGTGTGT GTGTGTGT |
| 27 | 728-745 | TGTGTGTGTG TGTGTGTG |
| 28 | 729-746 | GTGTGTGTGT GTGTGTGT |
| 29 | 730-747 | CGTGTGTGTG TGTGTGTG |
| 30 | 731-748 | GCGTGTGTGT GTGTGTGT |
| 31 | 732-749 | TGCGTGTGTG TGTGTGTG |
| 32 | 733-750 | GTGCGTGTGT GTGTGTGT |
| 33 | 971-988 | AGTGGCGGAT CTGAACTC |
| 34 | 972-989 | GAGTGGCGGA TCTGAACT |
| 35 | 1430-1447 | GAGTGTGCTG CAGGTAGA |
| 36 | 1432-1449 | TCGAGTGTGC TGCAGGTA |
| 37 | 1433-1450 | ATCGAGTGTG CTGCAGGT |
| 38 | 1570-1587 | GTGCTGTTGT AGATGGAA |
| 39 | 1571-1588 | GGTGCTGTTG TAGATGGA |
| 40 | 1572-1589 | TGGTGCTGTT GTAGATGG |
| 41 | 1573-1590 | CTGGTGCTGT TGTAGATG |
| 42 | 1574-1591 | CCTGGTGCTG TTGTAGAT |
| 43 | 1575-1592 | CCCTGGTGCT GTTGTAGA |
| 44 | 1576-1593 | TCCCTGGTGC TGTTGTAG |
| 45 | 1577-1594 | GTCCCTGGTG CTGTTGTA |
| 46 | 1578-1595 | AGTCCCTGGT GCTGTTGT |
| 47 | 1895-1912 | CTGGGTTGGA GATGTTAA |
| 48 | 1896-1913 | GCTGGGTTGG AGATGTTA |
| 49 | 1897-1914 | CGCTGGGTTG GAGATGTT |
| 50 | 1900-1917 | TAGCGCTGGG TTGGAGAT |
| 51 | 1903-1920 | ATGTAGCGCT GGGTTGGA |
| 52 | 1945-1962 | AGCCATTCGC CTTCTGCT |
| 53 | 1994-2011 | GTCTTTATGG TGAAGCCA |
| 54 | 1995-2012 | TGTCTTTATG GTGAAGCC |
| 55 | 1996-2013 | CTGTCTTTAT GGTGAAGC |
| 56 | 1997-2014 | CCTGTCTTTA TGGTGAAG |
| 57 | 2000-2017 | GTTCCTGTCT TTATGGTG |
| 58 | 2001-2018 | GGTTCCTGTC TTTATGGT |
| 59 | 2002-2019 | AGGTTCCTGT CTTTATGG |
| 60 | 2003-2020 | CAGGTTCCTG TCTTTATG |
| 61 | 2004-2021 | CCAGGTTCCT GTCTTTAT |
| 62 | 2183-2200 | GGTCTTCCCA CTGTTTTT |
| 63 | 2194-2211 | AGGAGATGTG GGGTCTTC |
| 64 | 2195-2212 | CAGGAGATGT GGGGTCTT |
| 65 | 2241-2258 | GGTTGGTCTG TTGTGACT |
| 66 | 2242-2259 | CGGTTGGTCT GTTGTGAC |
| 67 | 2243-2260 | CCGGTTGGTC TGTTGTGA |
| 68 | 2522-2539 | GAGAATGGTT AGAGGTTC |
| 69 | 2525-2542 | GTAGAGAATG GTTAGAGG |
| 70 | 2542-2559 | GGTGTTTTGC CAATGTAG |
| 71 | 2543-2560 | GGGTGTTTTG CCAATGTA |
| 72 | 2544-2561 | TGGGTGTTTT GCCAATGT |
| 73 | 2666-2683 | CATCATCGTT GTCGTCGT |
| 74 | 2979-2996 | GAACGGTACG TACAGCAA |
| 75 | 2980-2997 | GGAACGGTAC GTACAGCA |
| 76 | 2981-2998 | AGGAACGGTA CGTACAGC |
| 77 | 2982-2999 | TAGGAACGGT ACGTACAG |
| 78 | 2983-3000 | ATAGGAACGG TACGTACA |
| 79 | 2984-3001 | GATAGGAACG GTACGTAC |
| 80 | 2985-3002 | GGATAGGAAC GGTACGTA |
| 81 | 2986-3003 | GGGATAGGAA CGGTACGT |
| 82 | 3029-3046 | GGGTGCCTAT TGCATAGC |
| 83 | 3030-3047 | AGGGTGCCTA TTGCATAG |
| 84 | 3031-3048 | AAGGGTGCCT ATTGCATA |
| 85 | 3032-3049 | GAAGGGTGCC TATTGCAT |
| 86 | 3033-3050 | GGAAGGGTGC CTATTGCA |
| 87 | 3035-3052 | TGGGAAGGGT GCCTATTG |
| 88 | 3036-3053 | ATGGGAAGGG TGCCTATT |
| 89 | 3037-3054 | AATGGGAAGG GTGCCTAT |
| 90 | 3038-3055 | GAATGGGAAG GGTGCCTA |
| 91 | 3039-3056 | AGAATGGGAA GGGTGCCT |
| 92 | 3040-3057 | AAGAATGGGA AGGGTGCC |
| 93 | 3041-3058 | TAAGAATGGG AAGGGTGC |
| 94 | 3042-3059 | GTAAGAATGG GAAGGGTG |
| 95 | 3043-3060 | AGTAAGAATG GGAAGGGT |
| 96 | 3044-3061 | GAGTAAGAAT GGGAAGGG |
| 97 | 3259-3276 | CAGACTTTCT CGGTCATA |
| 98 | 3260-3277 | GCAGACTTTC TCGGTCAT |
| 99 | 3261-3278 | TGCAGACTTT CTCGGTCA |
| 100 | 3262-3279 | ATGCAGACTT TCTCGGTC |
| 101 | 3263-3280 | AATGCAGACT TTCTCGGT |
| 102 | 3264-3281 | TAATGCAGAC TTTCTCGG |
| 103 | 4281-4298 | GACCTGGACT TTTTTCCC |
| 104 | 4282-4299 | TGACCTGGAC TTTTTTCC |
| 105 | 4283-4300 | CTGACCTGGA CTTTTTTC |
| 106 | 4284-4301 | GCTGACCTGG ACTTTTTT |
| 107 | 4467-4484 | CTGCAATGAT GTGGCAAA |
| 108 | 4468-4485 | TCTGCAATGA TGTGGCAA |
| 109 | 4469-4486 | TTCTGCAATG ATGTGGCA |
| 110 | 4470-4487 | CTTCTGCAAT GATGTGGC |
| 111 | 5062-5079 | GCTGCCCACT TGCATACT |
| 112 | 5569-5586 | GTTGGCAGAA CATAGAAC |
| 113 | 5570-5587 | CGTTGGCAGA ACATAGAA |
| 114 | 5571-5588 | GCGTTGGCAG AACATAGA |
| 115 | 5616-5633 | ATGGGGCTAC AGGGGATA |
| 116 | 5617-5634 | TATGGGGCTA CAGGGGAT |
| 117 | 5618-5635 | TTATGGGGCT ACAGGGGA |
| 118 | 5620-5637 | AGTTATGGGG CTACAGGG |
| 119 | 5621-5638 | AAGTTATGGG GCTACAGG |
| 120 | 5622-5639 | CAAGTTATGG GGCTACAG |
| 121 | 5623-5640 | CCAAGTTATG GGGCTACA |
| 122 | 5624-5641 | TCCAAGTTAT GGGGCTAC |
| 123 | 5625-5642 | ATCCAAGTTA TGGGGCTA |
| 124 | 5626-5643 | TATCCAAGTT ATGGGGCT |
| 125 | 5627-5644 | CTATCCAAGT TATGGGGC |
| 126 | 5759-5776 | ATTGGAGGAA ATAGGGTG |
| 127 | 5783-5800 | GTCTTGTAGG TAGCAGCC |
| 128 | 5784-5801 | GGTCTTGTAG GTAGCAGC |
| 129 | 5785-5802 | TGGTCTTGTA GGTAGCAG |
| 130 | 5786-5803 | CTGGTCTTGT AGGTAGCA |
| 131 | 5787-5804 | TCTGGTCTTG TAGGTAGC |
| 132 | 5788-5805 | GTCTGGTCTT GTAGGTAG |
| 133 | 5789-5806 | AGTCTGGTCT TGTAGGTA |
| 134 | 5790-5807 | GAGTCTGGTC TTGTAGGT |
| 135 | 5791-5808 | GGAGTCTGGT CTTGTAGG |
| 136 | 5792-5809 | AGGAGTCTGG TCTTGTAG |

and chemically-modified variants thereof, LNA variants thereof, gapmer variants thereof, and any combination or pooling thereof.

12. The agent, use or method of claim 11, wherein the TGF-β2-specific antisense oligonucleotides have no more than one or two mismatches as compared to a target human TGF-β2.

13. The agent, use or method of claim 11, wherein the TGF-β2-specific antisense oligonucleotides reduce a TGF-β2 transcript level by at least 60%, or at least 70%, or at least 80%, or at least 90%.

14. The agent, use or method of claim 11, wherein the TGF-β2-specific antisense oligonucleotides reduce any TGF-β1 transcript level and any TGF-β3 transcript level by less than 10%, or less than 5%, or less than 1%.

15. The agent, use or method of claim 11, wherein the TGF-β2-specific antisense oligonucleotides have one or more nucleotides chemically modified as a phosphorothioate internucleoside linkage, a methoxypropylphosphonate internucleoside linkage, an aminophosphoro linkage to a morpholino group, a 2′-OMe ribose group, a 2′-MOE methoxyethyl ribose group, a 2′-4′ constrained methoxyethyl bicyclic ribose group, a 2′-4′ constrained ethyl bicyclic ribose group, an LNA ribose group, a 2′-F ribose group, or a 5-methylcytodine base.

16. The agent, use or method of claim 11, wherein the antisense agent is conjugated to a polyethylene glycol, a lipid, or a triantenarry N-acteyl-galactosamine.

17. The agent, use or method of any of claims 1-16, wherein the agent for inhibiting or suppressing expression of TGF-β2 comprises a carrier of sterile water for injection, saline, isotonic saline, or a combination thereof, which may be the same or different for each agent.

18. The agent, use or method of any of claims 1-17, wherein the agent for inhibiting or suppressing expression of TGF-β2 is substantially free of excipients.

19. The agent, use or method of any of claims 1-18, wherein the agent for inhibiting or suppressing expression of TGF-β2 is stable in a carrier substantially free of excipients for at least 14 days at 37° C.

20. The agent, use or method of any of claims 1-19, wherein patients are selected who have previously been treated with any agent comprising irinotecan.

21. The agent, use or method of any of claims 1-20, comprising using one or more biomarkers to select subjects who benefit from the agent, use or method.

22. The agent, use or method of any of claims 1-21, wherein patients are selected who have an elevated level of TGF-β2.

23. The agent, use or method of any of claims 1-22, wherein the one or more biomarkers are a level of a tumor mutation burden (TMB), a level of a tumor neoantigen, a level of clinical hypoxia, or a combination thereof.

24. The agent, use or method of any of claims 1-23, wherein the one or more biomarkers are a level of a tumor mutation burden, a level of a reduced neoantigen load determined in a tumor microenvironment, a level of a reduced macrophage determined in a tumor microenvironment, a level of a reduced mesenchymal stem cell MSC determined in a tumor microenvironment, a level of a type 2 T-helper cell Th2 determined in a tumor microenvironment, and a combination thereof.

25. The method, agent or use of any of claims 1-24, wherein the subject upon the administration or use has an improved level of at least one of the one or more biomarkers as compared to a level found in a healthy patient.

26. The agent, use or method of any of claims 1-25, wherein the administration or use decreases mortality rate at month 6, 12, 18, 24, 30, or 36.

27. The agent, use or method of any of claims 1-26, wherein the administration or use increases survival rate at month 6, 12, 18, 24, 30, or 36.

28. The agent, use or method of any of claims 1-27, wherein the administration or use increases health-related quality of life (HRQOL) by 15%, or 25%, or 35%, or 45%.

29. The agent, use or method of any of claims 1-28, in combination with any one or more medicaments comprising a targeted cancer drug, a cancer growth blocker, an EGFR inhibitor, and combinations thereof.

30. The agent, use or method of any of claims 1-29, in combination with any one or more medicaments selected from bevacizumab, everolimus, belzutifan, dabrafenib, trametinib, and combinations thereof.

31. The agent, use or method of any of claims 1-30, in combination with any one or more medicaments which are cancer growth blockers selected from an angiogenesis inhibitor, a histone deacetylase inhibitor, a hedgehog blocker, an mTOR inhibitor, a p53 inhibitor, a PARP inhibitor, a proteasome inhibitor, a tyrosine kinase inhibitor, and combinations thereof.

32. The agent, use or method of any of claims 1-31, in combination with any one or more medicaments which are EGFR inhibitors selected from erlotinib, gefitinib, afatinib, osimertinib, dacomitininb, and combinations thereof.

33. The agent, use or method of any of claims 1-32, in combination with a standard of care treatment for cancer.

34. The agent, use or method of any of claims 1-33, in combination with radiation therapy or electric field therapy.

35. A kit for treating or ameliorating the symptoms of cancer, the kit comprising:
a therapeutically effective amount of an antisense agent for inhibiting or suppressing expression of TGF-β2; and
a therapeutically effective amount of an agent comprising irinotecan.

36. The kit of claim 35, wherein the cancer is a pancreatic cancer, a colorectal cancer, a stomach cancer, a cervical cancer, a lung cancer, melanoma, a skin cancer, a breast cancer, a prostate cancer, a kidney cancer, an ovarian cancer, a thymus cancer, and multiple myeloma.

37. The kit of any of claims 35-36, wherein the agent comprising irinotecan is a FOLFIRINOX agent, a NALIRIFOX agent, an IRIFOX agent, or an IRINOX agent.

38. The kit of any of claims 35-37, wherein the agent comprising irinotecan comprises a nano-sized particle, a nano-sized micelle, a nano-sized liposome, or a nano-sized exosome.

39. The kit of any of claims 35-38, wherein the agent for inhibiting or suppressing expression of TGF-β2 and the agent comprising irinotecan are administered concurrently, simultaneously, sequentially, or separately in time.

40. The kit of any of claims 35-39, wherein the agent for inhibiting or suppressing expression of TGF-β2 and the agent comprising irinotecan are administered separately or in combination by injection or infusion.

41. The kit of any of claims 35-40, wherein the agent for inhibiting or suppressing expression of TGF-β2 is a TGF-β2-specific antisense oligonucleotide complementary to a TGF-β2 transcript and 15-30 nucleotides in length.

42. The kit of any of claims 35-41, wherein the agent for inhibiting or suppressing expression of TGF-β2 is a TGF-β2-specific antisense oligonucleotides complementary to a TGF-β2 pre-RNA, pre-mRNA or mRNA and 18-21 nucleotides in length.

43. The kit of any of claims 35-42, wherein the agent for inhibiting or suppressing expression of TGF-β2 is one or more TGF-β2-specific antisense oligonucleotides complementary to a TGF-β2 transcript as given in Table 1.

44. The kit of any of claims 35-43, wherein the TGF-β2-specific antisense oligonucleotides have no more than one or two mismatches as compared to a target human TGF-2.

45. The kit of any of claims 35-44, wherein the TGF-2-specific antisense oligonucleotides reduce a TGF-β2 transcript level by at least 60%, or at least 70%, or at least 80%, or at least 90%.

46. The kit of any of claims 35-45, wherein the TGF-β2-specific antisense oligonucleotides reduce any TGF-β1 transcript level and any TGF-β3 transcript level by less than 10%, or less than 5%, or less than 1%.

47. The kit of any of claims 35-46, wherein the TGF-β2-specific antisense oligonucleotides have one or more nucleotides chemically modified as a phosphorothioate internucleoside linkage, a methoxypropylphosphonate internucleoside linkage, an aminophosphoro linkage to a morpholino group, a 2′-OMe ribose group, a 2′-MOE methoxyethyl ribose group, a 2′-4′ constrained methoxyethyl bicyclic ribose group, a 2′-4′ constrained ethyl bicyclic ribose group, an LNA ribose group, a 2′-F ribose group, or a 5-methylcytodine base.

48. The kit of any of claims 35-47, wherein the antisense agent is conjugated to a polyethylene glycol, a lipid, or a triantenarry N-acteyl-galactosamine.

49. The kit of any of claims 35-48, wherein the agent for inhibiting or suppressing expression of TGF-β2 comprises a carrier of sterile water for injection, saline, isotonic saline, or a combination thereof, which may be the same or different for each agent.

50. The kit of any of claims 35-49, wherein the agent for inhibiting or suppressing expression of TGF-β2 is substantially free of excipients.

51. The kit of any of claims 35-50, wherein the agent for inhibiting or suppressing expression of TGF-β2 is stable in a carrier substantially free of excipients for at least 14 days at 37° C.


SEQUENCE LISTING (136 sequences)

SEQ ID NO: 1 - DNA, 18 bp, synthetic construct
  source 1..18: mol_type = other DNA
cggcatgtctattttgta

SEQ ID NO: 2 - DNA, 21 bp, synthetic construct
  source 1..21: mol_type = other DNA
taaagttcgtttagagaacag

SEQ ID NO: 3 - DNA, 15 bp, synthetic construct
  source 1..15: mol_type = other DNA
agccctgtatacgac

SEQ ID NO: 4 - DNA, 19 bp, synthetic construct
  source 1..19: mol_type = other DNA
gtaggtaaaaacctaatat

SEQ ID NO: 5 - DNA, 20 bp, synthetic construct
  source 1..20: mol_type = other DNA
cgtttagagaacagatctac

SEQ ID NO: 6 - DNA, 20 bp, synthetic construct
  source 1..20: mol_type = other DNA
cattgtagatgtcaaaagcc

SEQ ID NO: 7 - DNA, 21 bp, synthetic construct
  source 1..21: mol_type = other DNA
ctccctcatggtggcagttga

SEQ ID NO: 8 - DNA, 20 bp, synthetic construct
  source 1..20: mol_type = other DNA
gttcgtttagagaacagatc

SEQ ID NO: 9 - DNA, 18 bp, synthetic construct
  source 1..18: mol_type = other DNA
tttgttcctggatgactc

SEQ ID NO: 10 - DNA, 18 bp, synthetic construct
  source 1..18: mol_type = other DNA
gtttgttcctggatgact

SEQ ID NO: 11 - DNA, 18 bp, synthetic construct
  source 1..18: mol_type = other DNA
agtttgttcctggatgac

SEQ ID NO: 12 - DNA, 18 bp, synthetic construct
  source 1..18: mol_type = other DNA
cagtttgttcctggatga

SEQ ID NO: 13 - DNA, 18 bp, synthetic construct
  source 1..18: mol_type = other DNA
tcagtttgttcctggatg

SEQ ID NO: 14 - DNA, 18 bp, synthetic construct
  source 1..18: mol_type = other DNA
ctcagtttgttcctggat

SEQ ID NO: 15 - DNA, 18 bp, synthetic construct
  source 1..18: mol_type = other DNA
tgtgtgtgtgtgcgtgtg

SEQ ID NO: 16 - DNA, 18 bp, synthetic construct
  source 1..18: mol_type = other DNA
gtgtgtgtgtgtgcgtgt

SEQ ID NO: 17 - DNA, 18 bp, synthetic construct
  source 1..18: mol_type = other DNA
tgtgtgtgtgtgtgcgtg

SEQ ID NO: 18 - DNA, 18 bp, synthetic construct
  source 1..18: mol_type = other DNA
gtgtgtgtgtgtgtgcgt

SEQ ID NO: 19 - DNA, 18 bp, synthetic construct
  source 1..18: mol_type = other DNA
tgtgtgtgtgtgtgtgcg

SEQ ID NO: 20 - DNA, 18 bp, synthetic construct
  source 1..18: mol_type = other DNA
gtgtgtgtgtgtgtgtgc

SEQ ID NO: 21 - DNA, 18 bp, synthetic construct
  source 1..18: mol_type = other DNA
tgtgtgtgtgtgtgtgtg

SEQ ID NO: 22 - DNA, 18 bp, synthetic construct
  source 1..18: mol_type = other DNA
gtgtgtgtgtgtgtgtgt

SEQ ID NO: 23 - DNA, 18 bp, synthetic construct
  source 1..18: mol_type = other DNA
tgtgtgtgtgtgtgtgtg

SEQ ID NO: 24 - DNA, 18 bp, synthetic construct
  source 1..18: mol_type = other DNA
gtgtgtgtgtgtgtgtgt

SEQ ID NO: 25 - DNA, 18 bp, synthetic construct
  source 1..18: mol_type = other DNA
tgtgtgtgtgtgtgtgtg

SEQ ID NO: 26 - DNA, 18 bp, synthetic construct
  source 1..18: mol_type = other DNA
gtgtgtgtgtgtgtgtgt

SEQ ID NO: 27 - DNA, 18 bp, synthetic construct
  source 1..18: mol_type = other DNA
tgtgtgtgtgtgtgtgtg

SEQ ID NO: 28 - DNA, 18 bp, synthetic construct
  source 1..18: mol_type = other DNA
gtgtgtgtgtgtgtgtgt

SEQ ID NO: 29 - DNA, 18 bp, synthetic construct
  source 1..18: mol_type = other DNA
cgtgtgtgtgtgtgtgtg

SEQ ID NO: 30 - DNA, 18 bp, synthetic construct
  source 1..18: mol_type = other DNA
gcgtgtgtgtgtgtgtgt

SEQ ID NO: 31 - DNA, 18 bp, synthetic construct
  source 1..18: mol_type = other DNA
tgcgtgtgtgtgtgtgtg

SEQ ID NO: 32 - DNA, 18 bp, synthetic construct
  source 1..18: mol_type = other DNA
gtgcgtgtgtgtgtgtgt

SEQ ID NO: 33 - DNA, 18 bp, synthetic construct
  source 1..18: mol_type = other DNA
agtggcggatctgaactc

SEQ ID NO: 34 - DNA, 18 bp, synthetic construct
  source 1..18: mol_type = other DNA
gagtggcggatctgaact

SEQ ID NO: 35 - DNA, 18 bp, synthetic construct
  source 1..18: mol_type = other DNA
gagtgtgctgcaggtaga

SEQ ID NO: 36 - DNA, 18 bp, synthetic construct
  source 1..18: mol_type = other DNA
tcgagtgtgctgcaggta

SEQ ID NO: 37 - DNA, 18 bp, synthetic construct
  source 1..18: mol_type = other DNA
atcgagtgtgctgcaggt

SEQ ID NO: 38 - DNA, 18 bp, synthetic construct
  source 1..18: mol_type = other DNA
gtgctgttgtagatggaa

SEQ ID NO: 39 - DNA, 18 bp, synthetic construct
  source 1..18: mol_type = other DNA
ggtgctgttgtagatgga

SEQ ID NO: 40 - DNA, 18 bp, synthetic construct
  source 1..18: mol_type = other DNA
tggtgctgttgtagatgg

SEQ ID NO: 41 - DNA, 18 bp, synthetic construct
  source 1..18: mol_type = other DNA
ctggtgctgttgtagatg

SEQ ID NO: 42 - DNA, 18 bp, synthetic construct
  source 1..18: mol_type = other DNA
cctggtgctgttgtagat

SEQ ID NO: 43 - DNA, 18 bp, synthetic construct
  source 1..18: mol_type = other DNA
ccctggtgctgttgtaga

SEQ ID NO: 44 - DNA, 18 bp, synthetic construct
  source 1..18: mol_type = other DNA
tccctggtgctgttgtag

SEQ ID NO: 45 - DNA, 18 bp, synthetic construct
  source 1..18: mol_type = other DNA
gtccctggtgctgttgta

SEQ ID NO: 46 - DNA, 18 bp, synthetic construct
  source 1..18: mol_type = other DNA
agtccctggtgctgttgt

SEQ ID NO: 47 - DNA, 18 bp, synthetic construct
  source 1..18: mol_type = other DNA
ctgggttggagatgttaa

SEQ ID NO: 48 - DNA, 18 bp, synthetic construct
  source 1..18: mol_type = other DNA
gctgggttggagatgtta

SEQ ID NO: 49 - DNA, 18 bp, synthetic construct
  source 1..18: mol_type = other DNA
cgctgggttggagatgtt

SEQ ID NO: 50 - DNA, 18 bp, synthetic construct
  source 1..18: mol_type = other DNA
tagcgctgggttggagat

SEQ ID NO: 51 - DNA, 18 bp, synthetic construct
  source 1..18: mol_type = other DNA
atgtagcgctgggttgga

SEQ ID NO: 52 - DNA, 18 bp, synthetic construct
  source 1..18: mol_type = other DNA
agccattcgccttctgct

SEQ ID NO: 53 - DNA, 18 bp, synthetic construct
  source 1..18: mol_type = other DNA
gtctttatggtgaagcca

SEQ ID NO: 54 - DNA, 18 bp, synthetic construct
  source 1..18: mol_type = other DNA
tgtctttatggtgaagcc

SEQ ID NO: 55 - DNA, 18 bp, synthetic construct
  source 1..18: mol_type = other DNA
ctgtctttatggtgaagc

SEQ ID NO: 56 - DNA, 18 bp, synthetic construct
  source 1..18: mol_type = other DNA
cctgtctttatggtgaag

SEQ ID NO: 57 - DNA, 18 bp, synthetic construct
  source 1..18: mol_type = other DNA
gttcctgtctttatggtg

SEQ ID NO: 58 - DNA, 18 bp, synthetic construct
  source 1..18: mol_type = other DNA
ggttcctgtctttatggt

SEQ ID NO: 59 - DNA, 18 bp, synthetic construct
  source 1..18: mol_type = other DNA
aggttcctgtctttatgg

SEQ ID NO: 60 - DNA, 18 bp, synthetic construct
  source 1..18: mol_type = other DNA
caggttcctgtctttatg

SEQ ID NO: 61 - DNA, 18 bp, synthetic construct
  source 1..18: mol_type = other DNA
ccaggttcctgtctttat

SEQ ID NO: 62 - DNA, 18 bp, synthetic construct
  source 1..18: mol_type = other DNA
ggtcttcccactgttttt

SEQ ID NO: 63 - DNA, 18 bp, synthetic construct
  source 1..18: mol_type = other DNA
aggagatgtggggtcttc

SEQ ID NO: 64 - DNA, 18 bp, synthetic construct
  source 1..18: mol_type = other DNA
caggagatgtggggtctt

SEQ ID NO: 65 - DNA, 18 bp, synthetic construct
  source 1..18: mol_type = other DNA
ggttggtctgttgtgact

SEQ ID NO: 66 - DNA, 18 bp, synthetic construct
  source 1..18: mol_type = other DNA
cggttggtctgttgtgac

SEQ ID NO: 67 - DNA, 18 bp, synthetic construct
  source 1..18: mol_type = other DNA
ccggttggtctgttgtga

SEQ ID NO: 68 - DNA, 18 bp, synthetic construct
  source 1..18: mol_type = other DNA
gagaatggttagaggttc

SEQ ID NO: 69 - DNA, 18 bp, synthetic construct
  source 1..18: mol_type = other DNA
gtagagaatggttagagg

SEQ ID NO: 70 - DNA, 18 bp, synthetic construct
  source 1..18: mol_type = other DNA
ggtgttttgccaatgtag

SEQ ID NO: 71 - DNA, 18 bp, synthetic construct
  source 1..18: mol_type = other DNA
gggtgttttgccaatgta

SEQ ID NO: 72 - DNA, 18 bp, synthetic construct
  source 1..18: mol_type = other DNA
tgggtgttttgccaatgt

SEQ ID NO: 73 - DNA, 18 bp, synthetic construct
  source 1..18: mol_type = other DNA
catcatcgttgtcgtcgt

SEQ ID NO: 74 - DNA, 18 bp, synthetic construct
  source 1..18: mol_type = other DNA
gaacggtacgtacagcaa

SEQ ID NO: 75 - DNA, 18 bp, synthetic construct
  source 1..18: mol_type = other DNA
ggaacggtacgtacagca

SEQ ID NO: 76 - DNA, 18 bp, synthetic construct
  source 1..18: mol_type = other DNA
aggaacggtacgtacagc

SEQ ID NO: 77 - DNA, 18 bp, synthetic construct
  source 1..18: mol_type = other DNA
taggaacggtacgtacag

SEQ ID NO: 78 - DNA, 18 bp, synthetic construct
  source 1..18: mol_type = other DNA
ataggaacggtacgtaca

SEQ ID NO: 79 - DNA, 18 bp, synthetic construct
  source 1..18: mol_type = other DNA
gataggaacggtacgtac

SEQ ID NO: 80 - DNA, 18 bp, synthetic construct
  source 1..18: mol_type = other DNA
ggataggaacggtacgta

SEQ ID NO: 81 - DNA, 18 bp, synthetic construct
  source 1..18: mol_type = other DNA
gggataggaacggtacgt

SEQ ID NO: 82 - DNA, 18 bp, synthetic construct
  source 1..18: mol_type = other DNA
gggtgcctattgcatagc

SEQ ID NO: 83 - DNA, 18 bp, synthetic construct
  source 1..18: mol_type = other DNA
agggtgcctattgcatag

SEQ ID NO: 84 - DNA, 18 bp, synthetic construct
  source 1..18: mol_type = other DNA
aagggtgcctattgcata

SEQ ID NO: 85 - DNA, 18 bp, synthetic construct
  source 1..18: mol_type = other DNA
gaagggtgcctattgcat

SEQ ID NO: 86 - DNA, 18 bp, synthetic construct
  source 1..18: mol_type = other DNA
ggaagggtgcctattgca

SEQ ID NO: 87 - DNA, 18 bp, synthetic construct
  source 1..18: mol_type = other DNA
tgggaagggtgcctattg

SEQ ID NO: 88 - DNA, 18 bp, synthetic construct
  source 1..18: mol_type = other DNA
atgggaagggtgcctatt

SEQ ID NO: 89 - DNA, 18 bp, synthetic construct
  source 1..18: mol_type = other DNA
aatgggaagggtgcctat

SEQ ID NO: 90 - DNA, 18 bp, synthetic construct
  source 1..18: mol_type = other DNA
gaatgggaagggtgccta

SEQ ID NO: 91 - DNA, 18 bp, synthetic construct
  source 1..18: mol_type = other DNA
agaatgggaagggtgcct

SEQ ID NO: 92 - DNA, 18 bp, synthetic construct
  source 1..18: mol_type = other DNA
aagaatgggaagggtgcc

SEQ ID NO: 93 - DNA, 18 bp, synthetic construct
  source 1..18: mol_type = other DNA
taagaatgggaagggtgc

SEQ ID NO: 94 - DNA, 18 bp, synthetic construct
  source 1..18: mol_type = other DNA
gtaagaatgggaagggtg

SEQ ID NO: 95 - DNA, 18 bp, synthetic construct
  source 1..18: mol_type = other DNA
agtaagaatgggaagggt

SEQ ID NO: 96 - DNA, 18 bp, synthetic construct
  source 1..18: mol_type = other DNA
gagtaagaatgggaaggg

SEQ ID NO: 97 - DNA, 18 bp, synthetic construct
  source 1..18: mol_type = other DNA
cagactttctcggtcata

SEQ ID NO: 98 - DNA, 18 bp, synthetic construct
  source 1..18: mol_type = other DNA
gcagactttctcggtcat

SEQ ID NO: 99 - DNA, 18 bp, synthetic construct
  source 1..18: mol_type = other DNA
tgcagactttctcggtca

SEQ ID NO: 100 - DNA, 18 bp, synthetic construct
  source 1..18: mol_type = other DNA
atgcagactttctcggtc

SEQ ID NO: 101 - DNA, 18 bp, synthetic construct
  source 1..18: mol_type = other DNA
aatgcagactttctcggt

SEQ ID NO: 102 - DNA, 18 bp, synthetic construct
  source 1..18: mol_type = other DNA
taatgcagactttctcgg

SEQ ID NO: 103 - DNA, 18 bp, synthetic construct
  source 1..18: mol_type = other DNA
gacctggacttttttccc

SEQ ID NO: 104 - DNA, 18 bp, synthetic construct
  source 1..18: mol_type = other DNA
tgacctggacttttttcc

SEQ ID NO: 105 - DNA, 18 bp, synthetic construct
  source 1..18: mol_type = other DNA
ctgacctggacttttttc

SEQ ID NO: 106 - DNA, 18 bp, synthetic construct
  source 1..18: mol_type = other DNA
gctgacctggactttttt

SEQ ID NO: 107 - DNA, 18 bp, synthetic construct
  source 1..18: mol_type = other DNA
ctgcaatgatgtggcaaa

SEQ ID NO: 108 - DNA, 18 bp, synthetic construct
  source 1..18: mol_type = other DNA
tctgcaatgatgtggcaa

SEQ ID NO: 109 - DNA, 18 bp, synthetic construct
  source 1..18: mol_type = other DNA
ttctgcaatgatgtggca

SEQ ID NO: 110 - DNA, 18 bp, synthetic construct
  source 1..18: mol_type = other DNA
cttctgcaatgatgtggc

SEQ ID NO: 111 - DNA, 18 bp, synthetic construct
  source 1..18: mol_type = other DNA
gctgcccacttgcatact

SEQ ID NO: 112 - DNA, 18 bp, synthetic construct
  source 1..18: mol_type = other DNA
gttggcagaacatagaac

SEQ ID NO: 113 - DNA, 18 bp, synthetic construct
  source 1..18: mol_type = other DNA
cgttggcagaacatagaa

SEQ ID NO: 114 - DNA, 18 bp, synthetic construct
  source 1..18: mol_type = other DNA
gcgttggcagaacataga

SEQ ID NO: 115 - DNA, 18 bp, synthetic construct
  source 1..18: mol_type = other DNA
atggggctacaggggata

SEQ ID NO: 116 - DNA, 18 bp, synthetic construct
  source 1..18: mol_type = other DNA
tatggggctacaggggat

SEQ ID NO: 117 - DNA, 18 bp, synthetic construct
  source 1..18: mol_type = other DNA
ttatggggctacagggga

SEQ ID NO: 118 - DNA, 18 bp, synthetic construct
  source 1..18: mol_type = other DNA
agttatggggctacaggg

SEQ ID NO: 119 - DNA, 18 bp, synthetic construct
  source 1..18: mol_type = other DNA
aagttatggggctacagg

SEQ ID NO: 120 - DNA, 18 bp, synthetic construct
  source 1..18: mol_type = other DNA
caagttatggggctacag

SEQ ID NO: 121 - DNA, 18 bp, synthetic construct
  source 1..18: mol_type = other DNA
ccaagttatggggctaca

SEQ ID NO: 122 - DNA, 18 bp, synthetic construct
  source 1..18: mol_type = other DNA
tccaagttatggggctac

SEQ ID NO: 123 - DNA, 18 bp, synthetic construct
  source 1..18: mol_type = other DNA
atccaagttatggggcta

SEQ ID NO: 124 - DNA, 18 bp, synthetic construct
  source 1..18: mol_type = other DNA
tatccaagttatggggct

SEQ ID NO: 125 - DNA, 18 bp, synthetic construct
  source 1..18: mol_type = other DNA
ctatccaagttatggggc

SEQ ID NO: 126 - DNA, 18 bp, synthetic construct
  source 1..18: mol_type = other DNA
attggaggaaatagggtg

SEQ ID NO: 127 - DNA, 18 bp, synthetic construct
  source 1..18: mol_type = other DNA
gtcttgtaggtagcagcc

SEQ ID NO: 128 - DNA, 18 bp, synthetic construct
  source 1..18: mol_type = other DNA
ggtcttgtaggtagcagc

SEQ ID NO: 129 - DNA, 18 bp, synthetic construct
  source 1..18: mol_type = other DNA
tggtcttgtaggtagcag

SEQ ID NO: 130 - DNA, 18 bp, synthetic construct
  source 1..18: mol_type = other DNA
ctggtcttgtaggtagca

SEQ ID NO: 131 - DNA, 18 bp, synthetic construct
  source 1..18: mol_type = other DNA
tctggtcttgtaggtagc

SEQ ID NO: 132 - DNA, 18 bp, synthetic construct
  source 1..18: mol_type = other DNA
gtctggtcttgtaggtag

SEQ ID NO: 133 - DNA, 18 bp, synthetic construct
  source 1..18: mol_type = other DNA
agtctggtcttgtaggta

SEQ ID NO: 134 - DNA, 18 bp, synthetic construct
  source 1..18: mol_type = other DNA
gagtctggtcttgtaggt

SEQ ID NO: 135 - DNA, 18 bp, synthetic construct
  source 1..18: mol_type = other DNA
ggagtctggtcttgtagg

SEQ ID NO: 136 - DNA, 18 bp, synthetic construct
  source 1..18: mol_type = other DNA
aggagtctggtcttgtag